SEQUENCE LISTING (15 sequences)

SEQ ID NO: 1 - DNA, 1743 bp, Stenotrophomonas maltophilia
  gene (1)..(1743)
  CDS (1)..(1743)
atgtcccaggtaacgcaaccgcgtgtgcgtcgagtgtgggtggtccttggtgcgtccgttctgtcatcgctgctgctggccacgcctgcgctggccggtgatgtccagctcagcggcctgcagtccgcgccgacgcaccagcgtttcatcgtgaagtaccgcgacggcagcgccgccgtggccaacaccactgcgctggcttcgtcgctgaagacggccgccgctggcctggccagcagccagggccgcgcgctgggcctgcaggaggtccgcaagctggccgtcggcccgaccctggtcaagaccgaccgtccgctcgaccaggccgaatccgagctgctgatgcgcaagctggccgccgacccgaacgtggaatacgtcgaagtcgaccagatcatgcgcgcgacgctgaccccgaacgacacccgcttcagcgagcaatggggcttcggcacctccaatgccggcatcaacatccggccggcctgggacaaggccaccggcaccggcgtggtggtggccgtgatcgataccggcatcaccaaccatgccgatctcaacgccaacatcctgccgggctatgacttcatcagcgacgccgcgatggcgcgcgatggcggcggccgcgacaacaatccgaacgacgaaggcgactggtacggcgccaacgagtgtggctcgggcatcccggcctccaactccagctggcacggcacccacgttgccggcaccgtggccgcggtgaccaacaacagtaccggcgtggcaggtaccgcgttcaatgccaaggtcgtgccggtgcgcgtgctcggcaagtgcggcggctacacctccgacatcgccgatgcgatcgtgtgggcgtccggtggcaccgtcagcggcgtaccggccaacgcgaatccggccgaagtcatcaacctctcgctgggcggcggtggcagctgctcgaccacctaccagaacgcgatcaacggcgcggtcggccgtggcaccaccgtggtggtcgccgccggcaacagcaacaccaacgtgtcctcgtcggtgccggccaactgcccgaacgtgatcgcggtggcggcgaccacctcggccggtgcacgtgccagcttctccaactacggtaccggcatcgacatctcggcaccgggccagagcatcctgtccaccctcaacaccggcaccaccacgccgggcagcgcgacctacgcgtcgtacaacggcacctcgatggcggcgccgcacgtggccggcgtggtcgcgctgatgcagtcggtcgctccgagcccgctgagcccggcgcaggtcgagagcatcatcaagagcacggcacgtccgctgccgggcgcctgctcgggtggttgcggcgccggcatcatcgacgccaatgccgccgtggccgcggcgatcaacggtggcggcaacccgaacccgggtggcaacgtgctgcagaacaacgtgccggtgaccggcctgggcgctgcgaccggtgccgagctgaactacaccgtcgcggttccggccggcagcacccagctgcgcgtcgcgatcagtggcggcagcggtgatgccgacctgtatgtgcgccagggcagtgccccgaccgataccacctacacctgccgtccgtacctgagcggcaacagcgagacctgcaccatcaacagtcctgccgccggcacctggtatgtgcgggtgaaggcctacagcaccttctcgggcctgaccctcaacgcccagtactga

SEQ ID NO: 2 - protein, 580 aa, Stenotrophomonas maltophilia
MSQVTQPRVRRVWVVLGASVLSSLLLATPALAGDVQLSGLQSAPTHQRFIVKYRDGSAAVANTTALASSLKTAAAGLASSQGRALGLQEVRKLAVGPTLVKTDRPLDQAESELLMRKLAADPNVEYVEVDQIMRATLTPNDTRFSEQWGFGTSNAGINIRPAWDKATGTGVVVAVIDTGITNHADLNANILPGYDFISDAAMARDGGGRDNNPNDEGDWYGANECGSGIPASNSSWHGTHVAGTVAAVTNNSTGVAGTAFNAKVVPVRVLGKCGGYTSDIADAIVWASGGTVSGVPANANPAEVINLSLGGGGSCSTTYQNAINGAVGRGTTVVVAAGNSNTNVSSSVPANCPNVIAVAATTSAGARASFSNYGTGIDISAPGQSILSTLNTGTTTPGSATYASYNGTSMAAPHVAGVVALMQSVAPSPLSPAQVESIIKSTARPLPGACSGGCGAGIIDANAAVAAAINGGGNPNPGGNVLQNNVPVTGLGAATGAELNYTVAVPAGSTQLRVAISGGSGDADLYVRQGSAPTDTTYTCRPYLSGNSETCTINSPAAGTWYVRVKAYSTFSGLTLNAQY

SEQ ID NO: 3 - protein, 617 aa, Stenotrophomonas maltophilia
  misc_feature: StmPr1 (AJ291488), Windhorst et al., 2002
MIKKQNLRINVLAAAVLSMTAVGAVHAAGLPTREPVRQASAAQPGTDRIIVKYRAGSAAAGDRSAKLSTVQSALTRASLAGGTARASTLGPQVVRRLGVGADVIRLQGRLAPAELQRVLKELKADPAVQYAEADVKLRRSELRAGDVQPALAPNDPYYQQYQWHLHNATGGINAPSAWDVSQGEGVVVAVLDTGILPQHPDLVGNLLEGYDFISDAETSRRATNDRVPGAQDYGDWVENDNECYTGSVAEDSSWHGTHVAGTVAEQTNNGVGMAGVAHKAKVLPVRVLGKCGGYLSDIADAITWASGGTVAGVPANANPAEVINMSLGGSGSCDGTYQDAINGAISRGTTVVVAAGNETDNASKYRPASCDGVVTVGATRITGGITYYSNYGSRVDLSGPGGGGSVDGNPGGYVWQSGSDAATTPESGSYSYMGMGGTSMASPHVAAVAALVQSALIAKGKDPLAPAAMRTLLKETARPFPVSIPTATPIGTGIVDAKAALAKALEEPCTENCGPVATPLTNKTAVGGLNGTAGSSRLYSFEAAAGKQLSVITYGGTGNVSVYIAQGREPSASDNDGKSTRPGTSETVRVNKPVAGTYYIKVVGEAAYNGVSILATQ

SEQ ID NO: 4 - protein, 580 aa, Stenotrophomonas maltophilia
  misc_feature: StmPr2; AAP13815
MSQVTQPRVRRVWVVLGASVLSSLLLATPALAGDVHSAGLQSAPTHQRFIVKYRDGSAPVANTTALASSLKSAAAGLASSQGRALGLQEVRKLAVGPTLVRTDRPLDQAESELLMRKLAADPNVEYVEVDQIMRATLTPNDTRLSEQWGFGTSNASINVRPAWDKATGTGVVVAVIDTGITNHPDLNANILPGYDFISDAAMARDGGGRDNNPNDEGDWYGANECGSGIPASNSSWHGTHVAGTVAAVTNNSTGVAGTAFNAKVVPVRVLGKCGGYTSDIADAIVWASGGTVSGVPANANPAEVINMSLGGGGSCSTTYQNAINGAVSRGTTVVVAAGNSNTNVSSSVPANCPNVIAVAATTSAGARASFSNYGTGIDISAPGQSILSTLNTGTTTPGSASYASYNGTSMAAPHVAGVVALMQSVAPSPLSPAQVESIIKSTARPLPGACSGGCGAGIVDANAAVAAAINGGGNPNPGGNVLQNNVPVTGLGAATGAELNYTVAVPAGSSQLRVTISGGSGDADLYVRQGSAPTDTSYTCRPYLSGNSETCTINSPAAGTWYVRVKAYSTFSGLTLNAQY

SEQ ID NO: 5 - DNA, 1740 bp, Stenotrophomonas maltophilia
  misc_feature: Protease 1
atgtcccaggtaacgcaaccgcgtgtgcgtcgtgtgtgggtggtccttggtgcgtccgtcctgtcatcgctgctgctggccacgcctgcgctggccggtgatgtccagctcagcggcctgcagtccgcgccgacccaccagcgcttcatcgtgaagtaccgcgacggcagtgcgccggtggccaacaccaccgcgctggcctcttcgctgaagacggccgctgccggcatcgccagcagccagggccgcgcgctgggcctgcagcaggtccgcaggcttgccgtcggctccaccgtggtcaggaccgaccgtgcgctggaccaggccgaatccgagctgctgatgcgcaagctggccgccgacccgaacgtcgaatacgtcgaagtcgaccagatcatgcgcgcgacgctgacccccaacgacacccgtttcagcgagcaatggggcttcggtacgtccaacgcctcgatcaacgtgcgtccggcctgggacaaggccaccggtaccggcgtggtcgtggccgtgatcgataccggcatcaccaaccatgccgacctcaacgccaacatcctgcccggctatgacttcatcagcgacgcggcgatggcgcgcgatggcggtggccgtgacagcaatcccaacgatgagggtgactggtacggcgccaacgagtgcggctcgggcattccggcctccaactccagctggcatggcacccacgtggccggcacggtcgctgcggtgaccaacaacagcaccggcgtggccggtaccgccttcaacgccaaggtcgtgccggtgcgcgtgctcggcaagtgcggcggctacacctccgacatcgccgacgcgatcgtctgggcctccggtggcacggtcagcggtgtgccggccaatgccaatccggccgaggtcatcaacatgtcgctgggcggcggcggcagctgctcgagcacttaccagaacgcgatcaacggcgcggtcggccgcggcaccaccgtggtggtcgctgccggcaacagcaacaccaacgtctccagcgcggtaccggcgaactgcccgaacgtgatcgccgtggcggccaccacctcggcgggtgcgcgtgccagcttctccaactacggcaccggcatcgacatctccgcaccgggccagagcatcctgtccaccctcaacagcggcaccaccaccccgggcagcgccagctatgcgtcctacaacggcacctcgatggcggcaccgcacgtggcgggcgtggttgcactgatgcagtcggtggcaccgagcccgctcagcccggcgcaggtggaaagcatcatcaagagcaccgcgcgtccgctgccgggtgcgtgctcgggtggttgcggcgccggcatcatcgacgccgatgccgcggtgactgccgcgatcaacggcaccacgcccaacccgggtggcaccgtgctgcagaacaacgtgccggttaccggcctcggcgcagccagcggcgcatcgctcagctataccgtcaacgtcccggctggcagcacgcagctgcgcgtggcgatcagcggcggcagcggtgatgccgacctgtacctgcgccagggcagtgccccgaccgacaccgtctacacctgccgcccgtacctgagcggcaacaatgagacctgcaccgtcaacagccccaccgccggtacctggtacgtgcgggtgaaggcctacagcaccttctcgggcgtgaccctcaacgcccagtactga

SEQ ID NO: 6 - protein, 579 aa, Stenotrophomonas maltophilia
MSQVTQPRVRRVWVVLGASVLSSLLLATPALAGDVQLSGLQSAPTHQRFIVKYRDGSAPVANTTALASSLKTAAAGIASSQGRALGLQQVRRLAVGSTVVRTDRALDQAESELLMRKLAADPNVEYVEVDQIMRATLTPNDTRFSEQWGFGTSNASINVRPAWDKATGTGVVVAVIDTGITNHADLNANILPGYDFISDAAMARDGGGRDSNPNDEGDWYGANECGSGIPASNSSWHGTHVAGTVAAVTNNSTGVAGTAFNAKVVPVRVLGKCGGYTSDIADAIVWASGGTVSGVPANANPAEVINMSLGGGGSCSSTYQNAINGAVGRGTTVVVAAGNSNTNVSSAVPANCPNVIAVAATTSAGARASFSNYGTGIDISAPGQSILSTLNSGTTTPGSASYASYNGTSMAAPHVAGVVALMQSVAPSPLSPAQVESIIKSTARPLPGACSGGCGAGIIDADAAVTAAINGTTPNPGGTVLQNNVPVTGLGAASGASLSYTVNVPAGSTQLRVAISGGSGDADLYLRQGSAPTDTVYTCRPYLSGNNETCTVNSPTAGTWYVRVKAYSTFSGVTLNAQY

SEQ ID NO: 7 - DNA, 1740 bp, Stenotrophomonas maltophilia
  misc_feature: Protease 5
atgtcccaggtaacgcaaccgcgtgtgcgtcgtgtgtgggtggtccttggtgcgtccgttctgtcatcgctgctgctggccacgcctgcgctggccggtgatgtccagctcagcggtctgcagtccgcgccgacccatcagcggttcatcgtgaagtaccgcgacggcagtgcgccggtggccaacaccaccgcactcgcctcttcgctgaagaccgccgccgccggcatcgccagcagccagggccgcgcgctgggcctgcagcaggtccgcaagctggctgtcggcgccaccgtggtcaggaccgaccgcgcactggaccaggccgacgccgaactgctgatgcgcaagctcgccgccgacccgaacgtggaatacgtcgaagtcgaccagatcatgcgcgcgacgctgaccccgaacgacacccgcttcagcgagcagtggggcttcggtacgtccaacgcgtcgatcaacgtgcgcccggcctgggacaaggccaccggcaccggcgtggtcgtggccgtgatcgataccggcatcaccaaccatgccgacctcaatgccaacattcttcccggctacgacttcatcagcgacgcggcgatggcgcgcgatggcggtggccgtgacagcaatcccaacgacgagggcgactggtacggtgacaacgagtgtcaggccggttacccgggctcgaactccagctggcatggcacccacgtggccggcacggtcgctgcggtgaccaacaacagcaacggcgtggccggtaccgcattcaatgccaaggtcgtgccggtgcgcgtgctcggcaagtgcggcggctatacctccgacatcgccgatgccatcgtctgggcctccggcggcaccgtaagcggtgtgccggccaatgccaatccggccgaggtcatcaacctctcgctgggcggcggcggcagctgctcgaccacctaccagaacgccatcaacggcgcggtcggccgtggcaccaccgtggtggtcgctgccggcaacagcaacaccaacgtgtcctcgtcggtaccggcgaactgcccgaacgtgatcgcagtggccgcgaccacctcggcaggtgcacgtgccagcttctccaactacggcaccggcatcgacatctccgcaccgggccagggcatcctgtccaccctcaacagcggcaccaccaccccgggcagcgccagctacgcgtcctacaacggcacctcgatggcggccccgcacgtggccggcgtggtcgcgctgatgcagtcggtggccccgagcccgctcagcccggcgcaggtggaaagcatcatcaagagcaccgcgcgtccgctgccgggtgcgtgttcgggcggctgcggcgccggcatcatcgacgccgatgccgcagtgacagctgcgatcaacggcaccactcccaacccgggtggcaccgtgctgcagaacaacgtgccggtcaccggcctaggcgcggccagcggcgcatcgctcagctacaccgtcaacgtaccggctggcagcgcgcagctgcgcgtggcgatcagtggcggcagcggtgacgccgacctgtacctgcgccagggcagtgccccgaccgacaccgtctacacctgccgcccgtacctgagcggcaacaacgagacctgcaccgtcaacagccccgctgccggcgtatggcacgtgcgggtgaaggcgtacagcaccttctcaggcgtgaccctcaacgcccagtactga

SEQ ID NO: 8 - protein, 579 aa, Stenotrophomonas maltophilia
MSQVTQPRVRRVWVVLGASVLSSLLLATPALAGDVQLSGLQSAPTHQRFIVKYRDGSAPVANTTALASSLKTAAAGIASSQGRALGLQQVRKLAVGATVVRTDRALDQADAELLMRKLAADPNVEYVEVDQIMRATLTPNDTRFSEQWGFGTSNASINVRPAWDKATGTGVVVAVIDTGITNHADLNANILPGYDFISDAAMARDGGGRDSNPNDEGDWYGDNECQAGYPGSNSSWHGTHVAGTVAAVTNNSNGVAGTAFNAKVVPVRVLGKCGGYTSDIADAIVWASGGTVSGVPANANPAEVINLSLGGGGSCSTTYQNAINGAVGRGTTVVVAAGNSNTNVSSSVPANCPNVIAVAATTSAGARASFSNYGTGIDISAPGQGILSTLNSGTTTPGSASYASYNGTSMAAPHVAGVVALMQSVAPSPLSPAQVESIIKSTARPLPGACSGGCGAGIIDADAAVTAAINGTTPNPGGTVLQNNVPVTGLGAASGASLSYTVNVPAGSAQLRVAISGGSGDADLYLRQGSAPTDTVYTCRPYLSGNNETCTVNSPAAGVWHVRVKAYSTFSGVTLNAQY

SEQ ID NO: 9 - DNA, 1743 bp, Stenotrophomonas maltophilia
  misc_feature: Protease 7
atgtcccaggtaacgcaaccgcgtgtgcgtcgagtgtgggtggtccttggtgcgtccgttctgtcatcgctgctgctggccacgcctgcgctggccggtgatgtccagctcagcggcctgcagtccgcgccgacgcaccagcgcttcatcgtcaagtaccgcgacggcagcgccgccgtggccaacactactgcgctggcttcgtcgctgaagacggccgccgccggcctggccagcagccagggccgcgcgctgggcctgcagcaggtccgcaagctggccgtcggcccgaccctggtcaagaccgaccgtccgctcgaccaggccgaatccgagctgctgatgcgcaagctggccgccgacccgaacgtggaatacgtcgaagtcgaccagatcatgcgcgcgacgctgaccccgaacgacacccgcttcagcgagcaatggggcttcggcacctccaatgccggcatcaacatccggccggcctgggacaaggccaccggcaccggcgtggtggtggccgtgatcgataccggcatcaccaaccatgccgatctcaacgccaacatcctgccgggctatgacttcatcagcgacgccgcgatggcgcgcgatggcggcggccgcgacaacaatccgaacgacgaaggcgactggtacggcgccaacgagtgtggctcgggcatcccggcctccaactccagctggcacggcacccacgttgccggcaccgtggcggcggtgaccaacaacagtaccggcgtggcaggtaccgcgttcaatgccaaggtcgtgccggtgcgcgtgctcggcaagtgcggcggctacacctccgacatcgccgatgcgatcgtgtgggcgtccggtggcaccgtcagcggcgtaccggccaacgccaatccggccgaagtcatcaacctctcgctgggcggcggtggcagctgctcgaccacctaccagaacgcgatcaacggcgcggtcggccgtggcaccaccgtggtagtcgccgccggcaacagcaacaccaacgtgtcctcgtcggtgccggccaactgcccgaacgtgatcgcggtggcggcgaccacctcggccggtgcacgtgccagcttctccaactacggtaccggcatcgacatttcggcaccgggccagagcatcctctccaccctcaacaccggcaccaccacgccgggcagcgcgacctacgcgtcgtacaacggcacctcgatggcagcgccgcacgtggccggcgtggtcgcgctgatgcagtcggtcgctccgagcccgctgagcccggcgcaggtcgagagcatcatcaagagcacggcacgtccgctgccgggcgcctgctcgggtggttgcggcgccggcatcatcgacgccaatgccgccgtggccgcagcgatcaatggtggcggcaacccgaacccgggtggcaacgtgctgcagaacaacgtaccggtgaccggcctgggcgctgcgaccggtgccgagctgaactacaccgtcgcggttccggccggcagcacccagctgcgcgtcgcgatcagtggcggcagcggtgatgccgacctgtatgtgcgccaaggcagtgccccgaccgataccacctacacctgccgtccgtacctgagcggcaacagcgagacctgcaccatcaacagtcctgccgccggcacctggtatgtgcgggtgaaggcctacagcaccttctcgggcctgaccctcaacgcccagtactga

SEQ ID NO: 10 - protein, 580 aa, Stenotrophomonas maltophilia
MSQVTQPRVRRVWVVLGASVLSSLLLATPALAGDVQLSGLQSAPTHQRFIVKYRDGSAAVANTTALASSLKTAAAGLASSQGRALGLQQVRKLAVGPTLVKTDRPLDQAESELLMRKLAADPNVEYVEVDQIMRATLTPNDTRFSEQWGFGTSNAGINIRPAWDKATGTGVVVAVIDTGITNHADLNANILPGYDFISDAAMARDGGGRDNNPNDEGDWYGANECGSGIPASNSSWHGTHVAGTVAAVTNNSTGVAGTAFNAKVVPVRVLGKCGGYTSDIADAIVWASGGTVSGVPANANPAEVINLSLGGGGSCSTTYQNAINGAVGRGTTVVVAAGNSNTNVSSSVPANCPNVIAVAATTSAGARASFSNYGTGIDISAPGQSILSTLNTGTTTPGSATYASYNGTSMAAPHVAGVVALMQSVAPSPLSPAQVESIIKSTARPLPGACSGGCGAGIIDANAAVAAAINGGGNPNPGGNVLQNNVPVTGLGAATGAELNYTVAVPAGSTQLRVAISGGSGDADLYVRQGSAPTDTTYTCRPYLSGNSETCTINSPAAGTWYVRVKAYSTFSGLTLNAQY

SEQ ID NO: 11 - DNA, 1740 bp, Stenotrophomonas maltophilia
  misc_feature: Protease 10
atgtcccaggtaacgcaaccgcgtgtgcgtcgtgtgtgggtggtccttggtgcgtccgttctgtcatcgctgctgctggccacgcctgcgctggccggtgatgtccagctcagcggtctgcagtccgcgccgacccatcagcggttcatcgtgaagtaccgcgacggcagtgcgccggtggccaacaccaccgcactcgcctcttcgctgaagaccgccgccgccggcatcgccagcagccagggccgcgcgctgggcctgcagcaggtccgcaagctggctgtcggctccaccgtggtcaggaccgaccgcgcactggaccaggccgacgccgaactgctgatgcgcaagctcgccgccgacccgaacgtggaatacgtcgaagtcgaccagatcatgcgcgcgacgctgaccccgaacgacacccgcttcagcgagcagtggggcttcggtacgtccaacgcgtcgatcaacgtgcgcccggcctgggacaaggccaccggcaccggcgtggtcgtggccgtgatcgataccggcatcaccaaccatgccgacctcaatgccaacattcttcccggctacgacttcatcagcgacgcggcgatggcgcgcgatggcggtggccgtgacagcaatcccaacgacgagggcgactggtacggtgacaacgagtgtcaggccggttacccgggctcgaactccagctggcacggtacccacgtggccggcacggtcgctgcggtgaccaacaacagcaacggcgtggccggtaccgcattcaatgccaaggtcgtgccggtgcgcgtgctcggcaagtgcggcggctatacctccgacatcgccgatgccatcgtctgggcctccggcggcaccgtaagcggtgtgccggccaatgccaatccggccgaggtcatcaacctctcgctgggcggcggcggcagctgctcgaccacctaccagaacgccatcaacggcgcggtcggccgtggcaccaccgtggtggtcgctgccggcaacagcaacaccaacgtgtcctcgtcggtaccggcgaactgcccgaacgtgatcgcagtggccgcgaccacctcggcaggtgcacgtgccagcttctccaactacggcaccggcatcgacatctccgcaccgggccagggcatcctgtccaccctcaacagcggcaccaccaccccgggcagcgccagctacgcgtcctacaacggcacctcgatggcggccccgcacgtggccggcgtggtcgcgctgatgcagtcggtggccccgagcccgctcagcccggcgcaggtggaaagcatcatcaagagcaccgcgcgtccgctgccgggtgcgtgttcgggtggttgcggcgccggcatcatcgacgccgatgccgcagtgacagctgcgatcaacggcaccactcccaacccgggtggcaccgtgctgcagaacaacgtgccggtcaccggcctaggcgcggccagcggcgcatcgctcagctacaccgtcaacgtaccggctggcagcgcgcagctgcgcgtggcgatcagtggcggcagcggtgacgccgacctgtacctgcgccagggcagtgccccgaccgacaccgtctacacctgccgcccgtacctgagcggcaacaacgagacctgcaccgtcaacagccccgctgccggcgtatggcacgtgcgggtgaaggcgtacagcaccttctcgggcgtgaccctcaacgcccagtactga

SEQ ID NO: 12 - protein, 579 aa, Stenotrophomonas maltophilia
MSQVTQPRVRRVWVVLGASVLSSLLLATPALAGDVQLSGLQSAPTHQRFIVKYRDGSAPVANTTALASSLKTAAAGIASSQGRALGLQQVRKLAVGSTVVRTDRALDQADAELLMRKLAADPNVEYVEVDQIMRATLTPNDTRFSEQWGFGTSNASINVRPAWDKATGTGVVVAVIDTGITNHADLNANILPGYDFISDAAMARDGGGRDSNPNDEGDWYGDNECQAGYPGSNSSWHGTHVAGTVAAVTNNSNGVAGTAFNAKVVPVRVLGKCGGYTSDIADAIVWASGGTVSGVPANANPAEVINLSLGGGGSCSTTYQNAINGAVGRGTTVVVAAGNSNTNVSSSVPANCPNVIAVAATTSAGARASFSNYGTGIDISAPGQGILSTLNSGTTTPGSASYASYNGTSMAAPHVAGVVALMQSVAPSPLSPAQVESIIKSTARPLPGACSGGCGAGIIDADAAVTAAINGTTPNPGGTVLQNNVPVTGLGAASGASLSYTVNVPAGSAQLRVAISGGSGDADLYLRQGSAPTDTVYTCRPYLSGNNETCTVNSPAAGVWHVRVKAYSTFSGVTLNAQY

SEQ ID NO: 13 - DNA, 1740 bp, Stenotrophomonas maltophilia
  misc_feature: Protease 6
atgtcccaggtaacgcaaccgcgtgtgcgtcgtgtgtgggtggtccttggtgcgtccgttctgtcatcgctgctgctggccacgcctgcgctggccggtgatgtccagctcagcggtctgcagtccgcgccgacccatcagcggttcatcgtgaagtaccgcgacggcagtgcgccggtggccaacaccaccgcactcgcctcttcgctgaagaccgccgccgccggcatcgccagcagccagggccgcgcgctgggcctgcagcaggtccgcaagctggctgtcggctccaccgtggtcaggaccgaccgcgcactggaccaggccgacgccgaactgctgatgcgcaagctcgccgccgacccgaacgtggaatacgtcgaagtcgaccagatcatgcgcgcgacgctgaccccgaacgacacccgcttcagcgagcagtggggcttcggtacgtccaacgcgtcgatcaacgtgcgcccggcctgggacaaggccaccggcaccggcgtggtcgtggccgtgatcgataccggcatcaccaaccatgccgacctcaatgccaacattcttcccggctacgacttcatcagcgacgcggcgatggcgcgcgatggcggtggccgtgacagcaatcccaacgacgagggcgactggtacggtgacaacgagtgtcaggccggttacccgggctcgaactccagctggcatggcacccacgtggccggcacggtcgctgcggtgaccaacaacagcaacggcgtggccggtaccgcattcaatgccaaggtcgtgccggtgcgcgtgctcggcaagtgcggcggctatacctccgacatcgccgatgccatcgtctgggcctccggcggcaccgtaagcggtgtgccggccaatgccaatccggccgaggtcatcaacctctcgctgggcggcggcggcagctgctcgaccacctaccagaacgccatcaacggcgcggtcggccgtggcaccaccgtggtggtcgctgccggcaacagcaacaccaacgtgtcctcgtcggtaccggcgaactgcccgaacgtgatcgcagtggccgcgaccacctcggcaggtgcacgtgccagcttctccaactacggcaccggcatcgacatctccgcaccgggccagggcatcctgtccaccctcaacagcggcaccaccaccccgggcagcgccagctacgcgtcctacaacggcacctcgatggcggccccgcacgtggccggcgtggtcgcgctgatgcagtcggtggccccgagcccgctcagcccggcgcaggtggaaagcatcatcaagagcaccgcgcgtccgctgccgggtgcgtgttcgggtggttgcggcgccggcatcatcgacgccgatgccgcagtgacagctgcgatcaacggcaccactcccaacccgggtggcaccgtgctgcagaacaacgtgccggtcaccggcctaggcgcggccagcggcgcatcgctcagctacaccgtcaacgtaccggctggcagcgcgcagctgcgcgtggcgatcagtggcggcagcggtgacgccgacctgtacctgcgccagggcagtgccccgaccgacaccgtctacacctgccgcccgtacctgagcggcaacaacgagacctgcaccgtcaacagccccgctgccggcgtatggcacgtgcgggtgaaggcgtacagcaccttctcgggcgtgaccctcaacgcccagtactga

SEQ ID NO: 14 - protein, 579 aa, Stenotrophomonas maltophilia; identical to SEQ ID NO: 12

SEQ ID NO: 15 - protein, 275 aa, Bacillus sp. [sequence omitted]

(19) United States
(12) Patent Application Publication
(10) Pub. No.: US 2010/0234267 A1
(43) Pub. Date: Sep. 16, 2010

(54) AGENTS CONTAINING PROTEASES FROM STENOTROPHOMONAS MALTOPHILIA

(75) Inventors: Petra Siegert, Haan (DE); Susanne Wieland, Zons/Dormagen (DE); Karl-Heinz Maurer, Erkrath (DE); Cornelius Bessler, Duesseldorf (DE); Doris Ribitsch, Graz (AT); Sonja Heumann, Graz (AT); Georg Gübitz, Hart bei Graz (AT); Wolfgang Karl, Graz (AT); Peter Remler, Graz (AT); Helmut Schwab, Graz (AT); Gabriele Berg, Graz (AT); Jochen Gerlach, Munich (DE)

Correspondence Address: WOODCOCK WASHBURN LLP, CIRA CENTRE, 12TH FLOOR, 2929 ARCH STREET, PHILADELPHIA, PA, 19104-2891, US

(73) Assignee: HENKEL AG & CO. KGAA, Duesseldorf (DE)

(21) Appl. No.: 12/685,215

(22) Filed: Jan. 11, 2010

Related U.S. Application Data
(63) Continuation of application No. PCT/EP2008/058546, filed on Jul. 3, 2008.

(30) Foreign Application Priority Data
Jul. 13, 2007 (DE) 10 2007 033104.7

(51) Int. Cl. C11D 7/42; C12N 9/48

(52) U.S. Cl. 510/392; 435/212

(57) ABSTRACT

The present invention relates to agent compositions, in particular washing and cleaning agents, that contain a protease from a bacterial strain of the species Stenotrophomonas maltophilia, and to said proteases themselves. The invention further relates to cleaning methods in which said agents are used, and to uses of said agents. The invention further relates to the manufacture and use of these proteases themselves.

This application is a continuation of PCT application serial number PCT/EP2008/058546, filed Jul. 3, 2008, which claims the benefit of German patent application serial number 102007033104.7, filed Jul. 13, 2007, each of which is incorporated herein by reference in its entirety.

The present invention relates to agent compositions, in particular washing and cleaning agents, that contain a protease from a bacterial strain of the species Stenotrophomonas maltophilia, and to said proteases themselves. The invention further relates to cleaning methods in which said agents are used, and to uses of said agents. The invention further relates to the manufacture and use of these proteases themselves.

It is known from the existing art that bacterial strains of the species Stenotrophomonas maltophilia produce enzymes that possess a proteolytic activity.

For example, Vazquez et al. (Rev. Argent. Microbiol. 32(2):53-62), 2000), report that the two Stenotrophomonas maltophilia strains ANT-1-1 and ANT-7-1 express proteases and secrete those enzymes into the surrounding medium. It is further disclosed that the protease production of these strains is dependent on factors such as calcium concentration, pH of the culture medium, and others.

The publication of Windhorst et al. (Journal of Biological Chemistry Vol. 277, No. 13: 11042-11049, 2002) characterizes the predominantly present extracellular protease of a human-pathogenic Stenotrophomonas maltophilia strain of a patient at the Hamburg University Clinic. The sequence of this protease, with the designation StmPr1, has been deposited in a publicly accessible database (GenBank™/EBI Data Bank) under accession number AJ291488, and is appended to this Application as SEQ ID NO. 3.

The publications of Miyaji et al. (Lett. Appl. Microbiol. 41(3): 253-7, 2005) and of Dunne et al. (Microbiology 146(8): 2069-78, 2000) also describe Stenotrophomonas strains that express enzymes having proteolytic activity. The specific strains disclosed are Stenotrophomonas maltophilia S-1 and Stenotrophomonas maltophilia W81.

No agents, in particular no washing and cleaning agents, in which a protease from a bacterial strain of the species Stenotrophomonas maltophilia is used are known, however, from the existing art.

It is preferred to use proteases of the subtilisin type for washing and cleaning agents. Examples thereof are the subtilisins BPN′ and Carlsberg, protease PB92, subtilisins 147 and 309, the alkaline protease from Bacillus lentus, subtilisin DY, and the enzymes (to be classified, however, as subtilases rather than as subtilisins in the strict sense) thermitase, proteinase K, and proteases TW3 and TW7. Subtilisin Carlsberg is obtainable in further developed form under the trade name Alcalase® from Novozymes A/S, Bagsvaerd, Denmark. Subtilisins 147 and 309 are marketed by the Novozymes company under the trade names Esperase® and Savinase®, respectively. The variants marketed under the designation BLAP® are derived from the protease from Bacillus lentus DSM 5483 (WO 91/02792 A1) and are described in particular in WO 92/21760 A1, WO 95/23221 A1, WO 02/088340 A2, and WO 03/038082 A2. Further usable proteases from various Bacillus species and from B. gibsonii are evident from patent applications WO 03/054185 A1, WO 03/056017 A2, WO 03/055974 A2, and WO 03/054184 A1.

Other usable proteases are, for example, the enzymes obtainable under the trade names Durazym®, Relase®, Everlase®, Nafizym, Natalase®, Kannase®, and Ovozymes® from the Novozymes company, under the trade names Purafect®, Purafect® OxP, Purafect® Prime and Properase® from the Genencor company, under the trade name Protosol® from Advanced Biochemicals Ltd., Thane, India, under the trade name Wuxi® from Wuxi Snyder Bioproducts Ltd., China, under the trade names Proleather® and Protease P® from Amano Pharmaceuticals Ltd., Nagoya, Japan, and under the designation Proteinase K-16 from Kao Corp., Tokyo, Japan.

The proteases used in agents according to the present invention either derive originally from microorganisms, for example the genera Bacillus, Streptomyces, Humicola, or Pseudomonas, and/or are produced by suitable microorganisms in accordance with biotechnological methods known per se, for example by transgenic expression hosts of Bacillus species or filamentous fungi.

A disadvantage of these proteases of the existing art preferably used in washing and cleaning agents is that they do not possess satisfactory proteolytic activity especially at low temperatures, for example between 20° C. and 60° C., and therefore do not exhibit optimum cleaning performance in this temperature range, especially in washing agents and dishwashing agents.

The object on which the present invention is based is therefore that of making available agents that, at low temperatures, in particular in a temperature range between 20 and 60°, exhibit improved removal of protein-containing deposits with respect to at least one stain, by preference with respect to multiple stains.

A further object of the present invention is to make available protease enzymes of this same kind that are suitable for use in agents according to the present invention and are notable for good, preferably for improved, proteolytic activity with respect to at least one stain, by preference with respect to multiple stains, in the temperature range recited above, when they are used in agents according to the present invention.

This object is achieved according to the present invention, in accordance with the teaching of claim 1, by making available agents that contain a protease which is obtainable naturally from a bacterial strain of the species Stenotrophomonas maltophilia. It has been found, surprisingly, that proteases from Stenotrophomonas maltophilia strains are advantageously usable in agents according to the present invention, and enable in agents according to the present invention at low temperatures, in particular in a temperature range between 20 and 60° C., improved removal of protease-sensitive deposits, for example stains on textiles or dishware.

One subject of the invention is therefore constituted by agents containing a protease that exists naturally in a bacterial strain of the species Stenotrophomonas maltophilia and/or that encompasses an amino acid sequence that is at least 50% identical to the amino acid sequence indicated in SEQ ID NO. 2 or SEQ ID NO. 6 or SEQ ID NO. 8 or SEQ ID NO. 10 or SEQ ID NO. 12 in a segment of at least 100 continuously connected amino acid residues. In further embodiments of the invention, the agents contain a protease that encompasses an amino acid sequence that is at least 50% identical to the amino acid sequence indicated in SEQ ID NO. 2 or SEQ ID NO. 6 or SEQ ID NO. 8 or SEQ ID NO. 10 or SEQ ID NO. 12 in a segment of, with increasing preference, 150, 200, 250, 300, 350, 400, 450, 477, 500, 550, 578 continuously connected amino acid residues. In a further embodiment of the invention, the agents contain a protease that encompasses an amino acid sequence that is at least 50% identical to the amino acid sequence indicated in SEQ ID NO. 2 or SEQ ID NO. 6 or SEQ ID NO. 8 or SEQ ID NO. 10 or SEQ ID NO. 12.

The proteases and nucleic acids, or corresponding segments, described in the present Application preferably exist naturally in a bacterial strain of the species Stenotrophomonas maltophilia or in bacteria that belong to such a bacterial strain. They are therefore also obtainable from such bacteria.

“Exist naturally” means in this connection that the protease is an endobacterial protease that can be isolated from the bacterium. This therefore does not include, in particular, those proteases that have been introduced with the aid of genetic-engineering methods into a bacterial strain according to the present invention and are expressed by said strain in recombinant fashion. Proteases commonly known from the existing art that are produced with the aid of a bacterial strain according to the present invention—i.e. for which the bacterial strain of the species Stenotrophomonas maltophilia is merely the production organism based on the introduction into that organism, by means of genetic-engineering methods, of the gene coding for the protease—are therefore not a subject of the invention.

It is of essential importance when considering all the subjects of the invention that segments according to the present invention be taken into account, since the proteases are posttranslationally modified. In particular, they can encompass amino acid sequences such as, for example, one or more N-terminal signal peptides(s) (e.g. as a secretion signal) and/or one or more further propeptide(s) and/or one or more further domain(s) that are no longer present in the mature protein, i.e. the mature protease. Segments particularly preferred according to the present invention, in particular for utilization in agents, methods, and uses according to the present invention, are therefore those that impart proteolytic activity, i.e. that are proteolytically active. Also preferred are the segments according to the present invention the completely processed, i.e. mature, proteases formed by bacteria of the respective bacterial strain of the species Stenotrophomonas maltophilia. In addition to possible further modifications, for example, these no longer comprise an N-terminal signal peptide.

The conventional procedure for recovering the enzyme is known to one skilled in the art of enzyme technology, and consists in removing the microorganism-containing samples from, for example, natural habitats and culturing them under conditions regarded as suitable, for example in an alkaline medium. Enriched cultures of the microorganisms that contain the desired enzymes, i.e. in the present case the protease enzymes, which are active under the relevant conditions, are thereby obtained. Of these, the microorganisms having the highest-performance enzymes are then selected and purified, for example by plating out onto protein-containing agar plates and measuring the lysis haloes that form, and the relevant genes are cloned.

A procedure of this kind is described, for example, in the textbook “Alkalophilic Microorganisms. A New Microbial World,” by K. Horikoshi and T. Akiba (1982), Japan Scientific Societies Press, Springer-Verlag, New York, Heidelberg, Berlin, ISBN 0-387-10924-2, Chapter 2, pp. 9-26. WO 00/24882 A1 also, for example, discloses a method for producing a gene bank, which is obtained by the fact that microorganism-containing samples from any habitats, for example from the rumen, are cultured under the conditions of interest and thereby enriched, and nucleic acids of interest are isolated therefrom and cloned.

Alkaline proteases formed naturally, preferably microbially, are therefore already in use in washing and cleaning agents. According to Application WO 93/07276 A1, for example, the protease 164-A1 of Chemgen Corp., Gaithersburg, Md., USA, and Vista Chemical Company, Austin, Tex., USA, obtainable from Bacillus spec. 164-A1, is suitable for use in washing and cleaning agents. Other examples are the alkaline protease from Bacillus sp. PD138, NCIMB 40338 of Novozymes A/S, Bagsvaerd, Denmark, (WO 93/18140 A1), the proteinase K-16 of Kao Corp., Tokyo, Japan deriving from Bacillus sp. ferm. BP-3376 (U.S. Pat. No. 5,344,770) and, according to WO 96/25489 A1, (Procter & Gamble, Cincinnati, Ohio, USA), the protease from the psychrophilic organism Flavobacterium balustinum.

Further alkaline proteases that are formed by microorganisms that can be isolated from natural habitats are evident, for example, from the applications WO 03/054185 A1 (from Bacillus gibsonii (DSM 14391)), WO 03/056017 A2 (from Bacillus sp. (DSM 14390)), WO 03/055974 A2 (from Bacillus sp. (DSM 14392)), and WO 03/054184 A1 (from Bacillus gibsonii (DSM 14393)). All these Applications also disclose corresponding washing and cleaning agents containing these novel alkaline proteases.

Further embodiments of the invention are characterized in that the protease contained in the agents according to the present invention encompasses an amino acid sequence that is with increasing preference at least 55%, 60%, 65%, 70%, 75%, 80%, 85%, 90%, 95%, 95.5%, 96%, 96.5%, 97%, 97.5% 98%, 98.5%, 99%, 99.5%, and very particularly preferably 100%, identical to the amino acid sequence indicated in SEQ ID NO. 2 or SEQ ID NO. 6 or SEQ ID NO. 8 or SEQ ID NO. 10 or SEQ ID NO. 12 in a segment of at least 100 continuously connected amino acid residues. In further embodiments of the invention, the agents contain a protease that encompasses an amino acid sequence that is with increasing preference at least 55%, 60%, 65%, 70%, 75%, 80%, 85%, 90%, 95%, 95.5%, 96%, 96.5%, 97%, 97.5% 98%, 98.5%, 99%, 99.5%, and very particularly preferably 100%, identical to the amino acid sequence indicated in SEQ ID NO. 2 or SEQ ID NO. 6 or SEQ ID NO. 8 or SEQ ID NO. 10 or SEQ ID NO. 12 in a segment of, with increasing preference, 150, 200, 250, 300, 350, 400, 450, 477, 500, 550, 578 continuously connected amino acid residues. In a preferred embodiment, the segment is selected in such a way that its C-terminus corresponds to position 470 or position 471 or position 472 or position 473 or position 474 or position 475 or position 476 or position 477 or position 478 or position 479 or position 480, with reference to SEQ ID NO. 2. This means that in an alignment, the C-terminal amino acid of the segment according to the present invention is associated with the aforesaid position in SEQ ID NO. 2, wherein the C-terminal amino acid of the segment and the corresponding amino acid in SEQ ID NO. 2 do not need to be identical, but can be identical. What is relevant is the position-related association of the amino acids, not their conformity. In a further embodiment of the invention, the agents contain a protease that encompasses an amino acid sequence that with increasing preference is at least 55%, 60%, 65%, 70%, 75%, 80%, 85%, 90%, 95%, 95.5%, 96%, 96.5%, 97%, 97.5% 98%, 98.5%, 99%, 99.5%, and very particularly preferably 100%, identical to the amino acid sequence indicated in SEQ ID NO. 2 or SEQ ID NO. 6 or SEQ ID NO. 8 or SEQ ID NO. 10 or SEQ ID NO. 12.

In a further preferred embodiment of the invention, the agent is characterized in that the washing performance of the protease that is contained corresponds at least to that of a protease that encompasses an amino acid sequence that corresponds to the amino acid sequence indicated in SEQ ID NO. 12 or to the amino acid sequence indicated in SEQ ID NO. 12 in a segment of, with increasing preference, 100, 150, 200, 250, 300, 350, 400, 450, 477, 500, 550, 578 continuously connected amino acid residues, the washing performance being determined in a washing system that contains a washing agent at a dosing ratio of between 4.5 and 7.0 grams per liter of washing bath and contains the protease, the proteases to be compared being used in equal-activity fashion and the washing performance being determined with respect to a whole egg/carbon black or whole egg/pigment stain on cotton, in particular the 10N whole egg/pigment on cotton stain, by measuring the degree of whiteness of the washed textiles, the washing operation being performed for at least 30 minutes, optionally 60 minutes, at a temperature of 40° C., and the water having a water hardness of between 15.5 and 16.5° German hardness.

According to the present invention, the terms “whole egg/carbon black” and “whole egg/pigment” are to be considered, with regard to stains, to be equivalent and to correspond to one another. A suitable stain is, for example, the commercially available 10N stain (whole egg/pigment) of wfk Testgewebe GmbH (Christenfeld 10, D-41379 Brüggen-Bacht, Germany).

A preferred liquid washing agent for a washing system of this kind has the following composition (all indications in wt %): 0.3-0.5% xanthan gum, 0.2-0.4% antifoaming agent, 6-7% glycerol, 0.3-0.5% ethanol, 4-7% FAEOS (fatty alcohol ether sulfate), 24-28% nonionic surfactants, 1% boric acid, 1-2% sodium citrate (dihydrate), 2-4% soda, 14-16% coconut fatty acids, 0.5% HEDP (1-hydroxyethane-(1,1-diphosphonic acid)), 0-0.4% PVP (polyvinylpyrrolidone), 0.0.05% optical brightener, 0-0.001% dye, remainder demineralized water. The dosing ratio of the liquid washing agent is preferably between 4.5 and 5.5 grams per liter of washing bath, for example 4.9 grams per liter of washing bath. Washing preferably occurs in a pH range between pH 8 and pH 10.5, preferably between pH 8 and pH 9.

A preferred powdered washing agent for a washing system of this kind has the following composition (all indications in wt %): 10% linear alkylbenzenesulfonate (sodium salt), 1.5% C12-C18 fatty alcohol sulfate (sodium salt), 2.0% C12-C18 fatty alcohol with 7 EO, 20% sodium carbonate, 6.5% sodium hydrogencarbonate, 4.0% amorphous sodium disilicate, 17% sodium carbonate peroxohydrate, 4.0% TAED, 3.0% polyacrylate, 1.0% carboxymethyl cellulose, 1.0% phosphonate, 25% sodium sulfate, remainder: optionally foam inhibitors, optical brightener, scents, and if applicable water to make 100%. The dosing ratio of the liquid washing agent is preferably between 6.0 and 7.0 grams per liter of washing bath, for example 6.7 grams per liter of washing bath. Washing preferably occurs in a pH range between pH 9 and pH 11.

The degree of whiteness, i.e. the lightening of the stains, is preferably determined using optical measurement methods, preferably photometrically. A device suitable for this is, for example, the Minolta CM508d spectrometer. The devices used for measurement are usually first calibrated using a white standard, preferably a white standard supplied therewith.

Equal-activity utilization ensures that the respective enzymatic properties, i.e. for example the washing performance on specific stains, are compared even if there is some drifting apart of the ratio of active substance to total protein (the values for specific activity). It is generally the case that a low specific activity can be compensated for by adding a larger quantity of protein. Methods for determining protease activities are familiar to one skilled in the art of enzyme technology, and are applied by him or her on a routine basis. Such methods are disclosed, for example, in Tenside, Vol. 7 (1970), pp. 125-132. The protease activity is indicated by preference in protease units (PU). Suitable protease activities, for example, are 5 or 10 PU (protease units) per ml of washing bath. The protease activity is not, however, equal to zero.

In a further embodiment of the invention, the agent is characterized in that the agent is a washing agent, hand washing agent, rinsing agent, hand dishwashing agent, automatic dishwashing agent, cleaning agent, dental-prosthesis or contact-lens care agent, post-treatment rinsing agent, disinfecting agent, or an agent for the treatment of filter media, textiles, furs, paper, fleeces, or leather. The agent is preferably characterized in that it is a laundry washing agent or a dishwashing agent.

All types of agents, in particular mixtures, formulations, solutions, etc., whose usability is improved by the addition of an above-described protein according to the present invention, are hereby included in the range of protection of the present invention. This may refer, for example, depending on the utilization sector, to solid mixtures, for example powders having freeze-dried or encapsulated proteins, or to gelled or liquid agents. Preferred formulations contain, for example, buffer substances, stabilizers, reaction partners, and/or cofactors of the proteases and/or other ingredients synergistic with the proteases.

This is understood to refer, in particular, to agents for the utilization sectors discussed below. Further utilization sectors are evident from the existing art and are presented, for example, in the manual “Industrial enyzmes and their applications” by H. Uhlig, Wiley-Verlag, New York, 1998.

In preferred embodiments of the invention, an agent according to the present invention is characterized in that it is a washing agent, hand washing agent, rinsing agent, hand dishwashing agent, automatic dishwashing agent, cleaning agent, dental-prosthesis or contact-lens care agent, post-treatment rinsing agent, disinfecting agent, cosmetic agent, pharmaceutical agent, or an agent for the treatment of filter media, textiles, furs, paper, fleeces, or leather.

Agents that are washing or cleaning agents are assigned to this subject of the invention as a preferred embodiment.

The reason is that, as shown in the exemplifying embodiments of the present Application, it has been possible to demonstrate an increase in performance for washing and cleaning agents having a protease preferred according to the present invention as compared with a protease-free agent, and to achieve very good cleaning performance in terms of protease-sensitive stains.

Included in this subject of the invention are all conceivable types of cleaning agent, both concentrates and agents to be utilized undiluted, for use on a commercial scale, in a washing machine, or for hand laundering or hand cleaning. These include, for example, washing agents for textiles, carpets, or natural fibers, for which the term “washing agent” is used in accordance with the present invention. These also include, for example, dishwashing agents for automatic dishwashers or manual dishwashing agents or cleaners for hard surfaces such as metal, glass, porcelain, ceramic, tiles, stone, painted surfaces, plastics, wood, or leather, for which the term “cleaning agent” is used in accordance with the present invention.

An agent according to the present invention can be both an agent for large-scale consumers or industrial users and a product for individual consumers, all types of washing and cleaning agents established in the existing art likewise representing embodiments of the present invention. These include, for example, concentrates and agents to be utilized undiluted, for use on a commercial scale in a washing machine or for hand laundering or hand cleaning. Also included therein are, for example, washing agents for textiles, carpets, or natural fibers, for which the term “washing agent” is used in accordance with the present invention. Also included therein are, for example, dishwashing agents for automatic dishwashers or manual dishwashing agents or cleaners for hard surfaces such as metal, glass, porcelain, ceramic, tiles, stone, painted surfaces, plastics, wood, or leather, for which the term “cleaning agent” is used in accordance with the present invention.

Embodiments of the present invention encompass all established and/or all useful presentation forms. Included thereamong are, for example, solid, powdered, liquid, gelled, or pasty agents, which optionally may also be made up of multiple phases and may be present in compressed or uncompressed form. A further subject of the invention is therefore represented by agents which are characterized in that they are present as single-component systems. Such agents are preferably made up of one phase. Agents according to the present invention can, however, of course also be made up of multiple phases. A further subject of the invention is therefore represented by agents which are characterized in that they are subdivided into multiple components. Also included among the presentation forms according to the present invention are extrudates, granulates, tablets, or pouches, which may be present packaged both in large containers and in portions. An agent according to the present invention is preferably characterized in that it contains the protease in a quantity from 2 μg to 20 mg, by preference from 5 μg to 17.5 mg, particularly preferably from 20 μg to 15 mg, and very particularly preferably from 50 μg to 10 mg, per g of the agent.

The washing or cleaning agents according to the present invention, which can be present as, in particular, powdered solids, in recompressed particle form, or as homogeneous solutions or suspensions, can in principle contain, in addition to the active substance (a protease according to the present invention) used according to the present invention, all known ingredients that are usual in such agents, at least one further ingredient being present in the agent. The agents according to the present invention can contain, in particular, builder substances, surface-active surfactants, bleaching agents based on organic and/or inorganic peroxygen compounds, bleach activators, water-miscible organic solvents, enzymes, sequestering agents, electrolytes, pH regulators, and further adjuvants such as optical brighteners, anti-gray agents, foam regulators, and dyes and scents, as well as combinations thereof.

A combination of a protease according to the present invention with one or more further ingredient(s) of the agents proves to be particularly advantageous, since such an agent exhibits improved cleaning performance because of synergisms that result, in particular between the protease and the further ingredient. This means that the agent brings about improved removal of stains, for example protein-containing stains, either as compared with an agent that contains only one of the two components or also as compared with the expected cleaning performance of an agent having both components based on simply adding the respective individual contributions of those two components to the cleaning performance of the agent. Such a synergism is achieved in particular by combining a protease according to the present invention with one of the surfactants and/or builder substances and/or bleaching agents described below.

The agents according to the present invention can contain a surfactant or multiple surfactants; anionic surfactants, nonionic surfactants, and mixtures thereof, but also cationic, zwitterionic, and amphoteric surfactants, are possibilities.

Suitable nonionic surfactants are, in particular, alkyl glycosides and ethoxylation and/or propoxylation products of alkyl glycosides, or linear or branched alcohols each having 12 to 18 carbon atoms in the alkyl portion and 3 to 20, by preference 4 to 10, alkyl ether groups. Also usable are corresponding ethoxylation and/or propoxylation products of N-alkylamines, vicinal diols, fatty acid esters and fatty acid amides that correspond in terms of the alkyl portion to the aforesaid long-chain alcohol derivatives, and of alkylphenols having 5 to 12 carbon atoms in the alkyl residue.

The nonionic surfactants used are by preference alkoxylated, advantageously ethoxylated, in particular primary alcohols having by preference 8 to 18 carbon atoms and an average of 1 to 12 mol ethylene oxide (EO) per mol of alcohol, in which the alcohol residue can be linear or preferably methyl-branched in the 2-position, or can contain mixed linear and methyl-branched residues, such as those that are usually present in oxo alcohol residues. Particularly preferred, however, are alcohol ethoxylates having linear residues made up of alcohols of natural origin having 12 to 18 carbon atoms, e.g. from coconut, palm, tallow, or oleyl alcohol, and an average of 2 to 8 EO per mol of alcohol. Included among the preferred ethoxylated alcohols are, for example, C₁₂₋₁₄ alcohols with 3 EO or 4 EO, C₉₋₁₁ alcohols with 7 EO, C₁₃₋₁₅ alcohols with 3 EO, 5 EO, 7 EO, or 8 EO, C₁₂₋₁₈ alcohols with 3 EO, 5 EO, or 7 EO, and mixtures thereof, such as mixtures of C₁₂₋₁₄ alcohol with 3 EO and C₁₂₋₁₈ alcohol with 7 EO. The degrees of ethoxylation indicated represent statistical averages, which can be an integral or a fractional number for a specific product. Preferred alcohol ethoxylates exhibit a restricted distribution of homologs (narrow range ethoxylates, NRE). In addition to these nonionic surfactants, fatty alcohols with more than 12 EO can also be used. Examples of these are (tallow) fatty alcohols with 14 EO, 16 EO, 20 EO, 25 EO, 30 EO, or 40 EO. Especially in agents for use in automatic methods, it is usual to use extremely low-foaming compounds. These include, by preference, C₁₂-C₁₈ alkylpolyethylene glycol-polypropylene glycol ethers having respectively up to 8 mol ethylene oxide and propylene oxide units in the molecule. It is also, however, possible to use other known low-foaming nonionic surfactants such as, for example, C₁₂-C₁₈ alkylpolyethylene glycol-polybutylene glycol ethers having respectively up to 8 mol ethylene oxide and butylene oxide units in the molecule, as well as end-capped alkylpolyalkylene glycol mixed ethers. Also particularly preferred are the hydroxyl-group-containing alkoxylated alcohols such as those described in European Patent Application EP 0 300 305 (so-called hydroxy mixed ethers). Also included among the nonionic surfactants are alkyl glycosides of the general formula RO(G)ₓ, in which R denotes a primary straight-chain or methyl-branched, especially methyl-branched in the 2-position, aliphatic residue having 8 to 22, by preference 12 to 18 carbon atoms, and G denotes a glycose unit having 5 or 6 carbon atoms, by preference glucose. The degree of oligomerization x, which indicates the distribution of monoglycosides and oligoglycosides, is any number (which, as a magnitude to be determined analytically, can also assume fractional values) between 1 and 10; by preference, x is 1.2 to 1.4. Also suitable are polyhydroxy fatty acid amides of formula (III) in which R¹CO denotes an aliphatic acyl residue having 6 to 22 carbon atoms; R² denotes hydrogen, an alkyl or hydroxyalkyl residue having 1 to 4 carbon atoms; and [Z] denotes a linear or branched polyhydroxyalkyl residue having 3 to 10 carbon atoms and 3 to 10 hydroxyl groups:

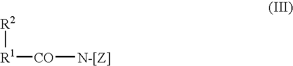

The polyhydroxy fatty acid amides are preferably derived from reducing sugars having 5 or 6 carbon atoms, in particular from glucose. Also belonging to the group of the polyhydroxy fatty acid amides are compounds of formula (IV)

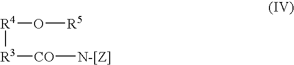

in which R³ denotes a linear or branched alkyl or alkenyl residue having 7 to 12 carbon atoms; R⁴ denotes a linear, branched, or cyclic alkylene residue or an arylene residue having 2 to 8 carbon atoms; and R⁵ denotes a linear, branched, or cyclic alkyl residue or an aryl residue or an oxyalkyl residue having 1 to 8 carbon atoms, C₁₋₄ alkyl or phenyl residues being preferred; and [Z] denotes a linear polyhydroxyalkyl residue whose alkyl chain is substituted with at least two hydroxyl groups, or alkoxylated, preferably ethoxylated or propoxylated, derivatives of that residue. Here as well, [Z] is preferably obtained by reductive amination of a sugar such as glucose, fructose, maltose, lactose, galactose, mannose, or xylose. The N-alkoxy- or N-aryloxy-substituted compounds can then be converted into the desired polyhydroxy fatty acid amides, for example by reaction with fatty acid methyl esters in the presence of an alkoxide as catalyst. A further class of nonionic surfactants used in preferred fashion, which are used either as the only nonionic surfactant or in combination with other nonionic surfactants, in particular together with alkoxylated fatty alcohols and/or alkyl glycosides, are alkoxylated, preferably ethoxylated or ethoxylated and propoxylated, fatty acid alkyl esters, by preference having 1 to 4 carbon atoms in the alkyl chain, in particular fatty acid methyl esters. Nonionic surfactants of the amine oxide type, for example N-cocalkyl-N,N-dimethylamine oxide and N-tallowalkyl-N,N-dihydroxyethylamine oxide, and the fatty acid alkanolamides, can also be suitable. The quantity of these nonionic surfactants is by preference no more than that of the ethoxylated fatty alcohols, in particular no more than half thereof. Further surfactants that are possible are so-called Gemini surfactants. These are understood in general as those compounds that possess two hydrophilic groups per molecule. These groups are usually separated from one another by a so-called “spacer.” This spacer is usually a carbon chain, which should be sufficiently long that the hydrophilic groups are far enough apart that they can act mutually independently. Surfactants of this kind are generally characterized by an unusually low critical micelle concentration, and by the ability to greatly reduce the surface tension of water. In exceptional cases the expression “Gemini surfactants” is understood to mean not only such dimeric, but also correspondingly “trimeric” surfactants. Suitable Gemini surfactants are, for example, sulfated hydroxy mixed ethers or dimeralcohol bis- and trimeralcohol tris-sulfates and ether sulfates. End-capped dimeric and trimeric mixed ethers are notable in particular for their bi- and multifunctionality. For example, the aforesaid end-capped surfactants possess good wetting properties and are also low-foaming, so that they are particularly suitable for use in automatic washing or cleaning methods. Gemini polyhydroxy fatty acid amides or polypolyhydroxy fatty acid amides can, however, also be used. The sulfuric acid monoesters of straight-chain or branched C₇₋₂₁ alcohols ethoxylated with 1 to 6 mol ethylene oxide, such as 2-methyl-branched C₉₋₁₁ alcohols with an average of 3.5 mol ethylene oxide (EO) or C₁₂₋₁₈ fatty alcohols with 1 to 4 EO, are also suitable. Also included among the preferred anionic surfactants are the salts of alkylsulfosuccinic acid, which are also referred to as sulfosuccinates or as sulfosuccinic acid esters and represent the monoesters and/or diesters of sulfosuccinic acid with alcohols, preferably fatty alcohols and in particular ethoxylated fatty alcohols. Preferred sulfosuccinates contain C₈ to C₁₈ fatty alcohol residues or mixtures thereof. Particularly preferred sulfosuccinates contain a fatty alcohol residue that is derived from ethoxylated fatty alcohols that, considered per se, represent nonionic surfactants. Sulfosuccinates whose fatty alcohol residues derive from ethoxylated fatty alcohols having a restricted homolog distribution are, in turn, particularly preferred. It is likewise also possible to use alk(en)ylsuccinic acid having by preference 8 to 18 carbon atoms in the alk(en)yl chain, or salts thereof. Further possible anionic surfactants are fatty acid derivatives of amino acids, for example of N-methyltaurine (taurides) and/or of N-methyglycine (sarcosides). Particularly preferred in this context are the sarcosides or sarcosinates, and here especially sarcosinates of higher and, if applicable, mono- or polyunsaturated fatty acids, such as oleyl sarcosinate. Further appropriate anionic surfactants are, in particular, soaps. Saturated fatty acid soaps, such as the salts of lauric acid, myristic acid, palmitic acid, stearic acid, hydrogenated erucic acid, and behenic acid, are suitable in particular, as are soap mixtures derived in particular from natural fatty acids, e.g. coconut, palm-kernel, or tallow fatty acids. The known alkenylsuccinic acid salts can also be used, together with these soaps or as a substitute agent for soaps.

The anionic surfactants, including the soaps, can be present in the form of their sodium, potassium, or ammonium salts and as soluble salts of organic bases such as mono-, di-, or triethanolamine. The anionic surfactants are preferably present in the form of their sodium or potassium salts, in particular in the form of the sodium salts.

Surfactants are contained in agents according to the present invention in quantitative proportions of, by preference, 5 wt % to 50 wt %, in particular from 8 wt % to 30 wt %.

An agent according to the present invention preferably contains at least one water-soluble and/or water-insoluble, organic and/or inorganic builder. Included among the water-soluble organic builder substances are polycarboxylic acids, in particular citric acid and sugar acids, monomeric and polymeric aminopolycarboxylic acids, in particular methylglycinediacetic acid, nitrilotriacetic acid, and ethylenediaminetetraacetic acid, as well as polyaspartic acid, polyphosphonic acids, in particular aminotris(methylenephosphonic acid), ethylenediaminetetrakis(methylenephosphonic acid), and 1-hydroxyethane-1,1-diphosphonic acid, polymeric hydroxy compounds such as dextrin, and (poly)carboxylic acids, in particular the polycarboxylates, accessible by the oxidation of polysaccharides or dextrins, polymeric acrylic acids, methacrylic acids, maleic acids, and mixed polymers thereof, which can also contain, polymerized into them, small concentrations of polymerizable substances without carboxylic-acid functionality. The relative molecular weight of the homopolymers of unsaturated carboxylic acids is generally between 3000 and 200,000, that of the copolymers between 2000 and 200,000, by preference 30,000 to 120,000, based in each case on free acid. A particularly preferred acrylic acid/maleic acid copolymer has a relative molecular weight from 30,000 to 100,000. Commercially usual products are, for example, Sokalan® CP 5, CP 10, and PA 30 of the BASF company. Suitable (although less preferred) compounds of this class are copolymers of acrylic acid or methacrylic acid with vinyl ethers, such as vinylmethyl ethers, vinyl esters, ethylene, propylene, and styrene, in which the proportion of acid is at least 50 wt %. It is also possible to use, as water-soluble organic builder substances, terpolymers that contain two unsaturated acids and/or salts thereof as monomers and, as a third monomer, vinyl alcohol and/or an esterified vinyl alcohol or a carbohydrate. The first acid monomer or its salt is derived from an ethylenically monounsaturated C₃ to C₈ carboxylic acid and by preference from a C₃ to C₄ monocarboxylic acid, in particular from (meth)acrylic acid. The second acid monomer or its salt can be a derivative of a C₄ to C₈ dicarboxylic acid (maleic acid being particularly preferred) and/or a derivative of an allylsulfonic acid that is substituted in the 2-position with an alkyl or aryl residue. Such polymers generally have a relative molecular weight between 1000 and 2,000,000. Further preferred copolymers are those that comprise, as monomers, by preference acrolein and acrylic acid/acrylic acid salts, or vinyl acetate. In particular for the manufacture of liquid agents, the organic builder substances can be used in the form of aqueous solutions, by preference in the form of 30- to 50-weight-percent aqueous solutions. All the aforesaid acids are generally used in the form of their water-soluble salts, in particular their alkali salts.

Organic builder substances of this kind can be contained, if desired, in quantities of up to 40 wt %, in particular up to 25 wt %, and by preference from 1 wt % to 8 wt %. Quantities close to the aforementioned upper limit are used by preference in pasty or liquid, in particular hydrous, agents according to the present invention.

Suitable water-soluble inorganic builder materials are, in particular, alkali silicates, alkali carbonates, and alkali phosphates, which can be present in the form of their alkaline, neutral, or acid sodium or potassium salts. Examples thereof are trisodium phosphate, tetrasodium diphosphate, disodium dihydrogendiphosphate, pentasodium triphosphate, so-called sodium hexametaphosphate, oligomeric trisodium phosphate having degrees of oligomerization from 5 to 1000, in particular 5 to 50, and the corresponding potassium salts, or mixtures of sodium and potassium salts. Crystalline or amorphous alkali aluminosilicates are used in particular as water-insoluble, water-dispersible inorganic builder materials, in quantities of up to 50 wt %, by preference not above 40 wt %, and in liquid agents in particular from 1 wt % to 5 wt %. Among these, the crystalline sodium aluminosilicates of washing-agent quality, in particular zeolite A, P, and if applicable X, alone or in mixtures, for example in the form of a co-crystal of zeolites A and X (Vegobond® AX, a commercial product of Condea Augusta S.p.A.) are preferred. Quantities close to the aforesaid upper limit are used by preference in solid, particulate agents. Suitable aluminosilicates exhibit, in particular, no particles having a particle size greater than 30 μm, and by preference are made up of at least 80 wt % particles having a size less than 10 μm. Their calcium binding capability, which can be determined as indicated in German Patent DE 24 12 837, is generally in the range from 100 to 200 mg CaO per gram.

Suitable substitutes or partial substitutes for the aforesaid aluminosilicate are crystalline alkali silicates, which can be present alone or mixed with amorphous silicates. The alkali silicates usable in the agents according to the present invention as detergency builders have by preference a molar ratio of alkali oxide to SiO₂ below 0.95, in particular from 1:1.1 to 1:12, and can be present in amorphous or crystalline fashion. Preferred alkali silicates are the sodium silicates, in particular the amorphous sodium silicates, having a Na₂O:SiO₂ molar ratio from 1:2 to 1:2.8. Crystalline sheet silicates of the general formula Na₂SiₓO₂ₓ₊₁.yH₂O, in which x, the so-called modulus, is a number from 1.9 to 22, in particular 1.9 to 4, and y is a number from 0 to 33, and preferred values for x are 2, 3, or 4, are preferred for use as crystalline silicates, which can be present alone or mixed with amorphous silicates. Preferred crystalline sheet silicates are those in which x in the aforesaid general formula assumes the values 2 or 3. In particular, both ®- and ™-sodium disilicates (Na₂Si₂O₅.yH₂O) are particularly preferred. Practically anhydrous crystalline alkali silicates manufactured from amorphous alkali silicates and having the aforesaid general formula, in which x denotes a number from 1.9 to 2.1, can be used in agents according to the present invention. In a further preferred embodiment of agents according to the present invention, a crystalline sodium sheet-form silicate having a modulus from 2 to 3 can be used, such as the one that can be manufactured from sand and soda. Crystalline sodium silicates having a modulus in the range from 1.9 to 3.5 are used in a further preferred embodiment of agents according to the present invention. Crystalline sheet-form silicates of formula (I) indicated above are marketed by Clariant GmbH under the trade name Na-SKS, e.g. Na-SKS-1 (Na₂Si₂₂O₄₅.xH₂O, kenyaite), Na-SKS-2 (Na₂Si₁₄O₂₉.xH₂O, magadiite), Na-SKS-3 (Na₂Si₈O₁₇.xH₂O), or Na-SKS-4 (Na₂Si₄O₉.xH₂O, makatite). Particularly suitable among these are Na-SKS-5 (<—Na₂Si₂O₅), Na-SKS-7 (β-Na₂Si₂O₅, natrosilite), Na-SKS-9 (NaHSi₂O₅.3H₂O), Na-SKS-10 (NaHSi₂O₅.3H₂O, kanemite), Na-SKS-11 (t-Na₂Si₂O₅), and Na-SKS-13 (NaHSi₂O₅), but in particular Na-SKS-6 (™-Na₂Si₂O₅). In a preferred embodiment of agents according to the present invention, a granular compound of crystalline sheet-form silicate and citrate, of crystalline sheet-form silicate and aforementioned (co)polymeric polycarboxylic acid, or of alkali silicate and alkali carbonate is used, for example as obtainable commercially under the name Nabion® 15.

Builder substances are contained in the agents according to the present invention by preference in quantities of up to 75 wt %, in particular 5 wt % to 50.

Peroxygen compounds that are suitable for use in agents according to the present invention are, in particular, organic peracids or peracid salts of organic acids such as phthalimidopercapronic acid, perbenzoic acid, or salts of diperdodecanedioic acid, hydrogen peroxide, and inorganic salts that release hydrogen peroxide under washing conditions, such as perborate, percarbonate, persilicate, and/or persulfate such as caroate. If solid peroxygen compounds are to be used, they can be utilized in the form of powders or granulates, which in principle can also be encased in known fashion. If an agent according to the present invention contains peroxygen compounds, they are present in quantities of preferably up to 50 wt %, in particular from 5 wt % to 30 wt %. The addition of small quantities of known bleaching-agent stabilizers, for example phosphonates, borates or metaborates, and metasilicates, as well as magnesium salts such as magnesium sulfate, may be useful.

Compounds that, under perhydrolysis conditions, yield aliphatic peroxocarboxylic acids having preferably 1 to 10 carbon atoms, in particular 2 to 4 carbon atoms, and/or (optionally substituted) perbenzoic acid, can be used as bleach activators. Substances that carry the O- and/or N-acyl groups having the aforesaid number of carbon atoms, and/or optionally substituted benzoyl groups, are suitable. Multiply acylated alkylenediamines, in particular tetraacetylethylendiamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl or isononanoyl oxybenzenesulfonate (n- or iso-NOBS), carboxylic acid anhydrides, in particular phthalic acid anhydride, acylated polyvalent alcohols, in particular triacetin, ethylene glycol diacetate, 2,5-diacetoxy-2,5-dihydrofuran, and enol esters, as well as acetylated sorbitol and mannitol and described mixtures thereof (SORMAN), acylated sugar derivatives, in particular pentaacetylglucose (PAG), pentaacetylfructose, tetraacetylxylose and octaacetyllactose, as well as acetylated, optionally N-alkylated glucamine and gluconolactone, and/or N-acylated lactams, for example N-benzoylcaprolactam, are preferred. The hydrophilically substituted acyl acetates and acyl lactams are also used in preferred fashion. Combinations of conventional bleach activators can also be used. Especially when aforesaid hydrogen peroxide-yielding bleaching agents are present, such bleach activators can be contained in the usual quantity range, by preference in quantities from 0.5 wt % to 10 wt %, in particular 1 wt % to 8 wt %, based on the entire agent; but they are by preference entirely absent when percarboxylic acid is used as the only bleaching agent.

In addition to or instead of the conventional bleach activators, sulfonimines and/or bleach-intensifying transition-metal salts or transition-metal complexes can also be contained as so-called bleach catalysts.

Included among the organic solvents (alongside water) usable in the agents according to the present invention, especially when the latter exist in liquid or pasty form, are alcohols having 1 to 4 carbon atoms, in particular methanol, ethanol, isopropanol, and tert.-butanol, diols having 2 to 4 carbon atoms, in particular ethylene glycol and propylene glycol, and mixtures thereof, and the ethers derivable from the aforesaid compound classes. Water-miscible solvents of this kind are present in the agents according to the present invention by preference in quantities not above 30 wt %, in particular from 6 wt % to 20 wt %.

In order to establish a desired pH that does not result spontaneously from mixture of the other components, the agents according to the present invention can contain system-compatible and environmentally compatible acids, in particular citric acid, acetic acid, tartaric acid, malic acid, lactic acid, glycolic acid, succinic acid, glutaric acid, and/or adipic acid, but also mineral acids, in particular sulfuric acid, or bases, in particular ammonium or alkali hydroxides. pH regulators of this kind are contained in the agents according to the present invention in quantities preferably not above 20 wt %, in particular from 1.2 wt % to 17 wt/o.

The purpose of anti-gray agents is to keep dirt that has been detached from the fibers suspended in the washing bath. Water-soluble colloids, usually organic in nature, are suitable for this, for example starch, size, gelatin, salts of ethercarboxylic acids or ethersulfonic acids of starch or of cellulose, or salts of acid sulfuric acid esters of cellulose or of starch. Water-soluble polyamides containing acid groups are also suitable for this purpose. Starch products other than those cited above can also be used, for example aldehyde starches. It is preferred to use cellulose ethers such as carboxymethyl cellulose (Na salt), methyl cellulose, hydroxyalkyl cellulose, and mixed ethers such as methylhydroxyethyl cellulose, methylhydroxypropyl cellulose, methylcarboxymethyl cellulose, and mixtures thereof, for example in quantities from 0.1 to 5 wt % based on the agent.

Textile washing agents according to the present invention can contain as optical brighteners, for example, derivatives of diaminostilbenedisulfonic acid or its alkali metal salts, although for use as color washing agents they are preferably free of optical brighteners. Suitable, for example, are salts of 4,4′-bis(2-anilino-4-morpholino-1,3,5-triazinyl-6-amino)stilbene-2,2′-disulfonic acid, or compounds of similar structure that carry, instead of the morpholino group, a diethanolamino group, a methylamino group, an anilino group, or a 2-methoxyethylamino group. Brighteners of the substituted diphenylstyryl type can also be present, e.g. the alkali salts of 4,4′-bis(2-sulfostyryl)diphenyl, of 4,4′-bis(4-chloro-3-sulfostyryl)diphenyl, or of 4-(4-chlorostyryl)-4′-(2-sulfostyryl)diphenyl. Mixtures of the aforesaid optical brighteners can also be used.

For use in automatic washing methods in particular, it can be advantageous to add usual foam inhibitors to the agents. Suitable as foam inhibitors are, for example, soaps of natural or synthetic origin that have a high proportion of C₁₈ to C₂₄ fatty acids. Suitable non-surfactant foam inhibitors are, for example, organopolysiloxanes and mixtures thereof with microfine, optionally silanated silicic acid, as well as paraffins, waxes, microcrystalline waxes, and mixtures thereof with silanated silicic acid or bistearylalkylenediamides. Mixtures of different foam inhibitors, for example those made of silicones, paraffins, or waxes, are also used with advantage. The foam inhibitors, in particular silicone- and/or paraffin-containing foam inhibitors, are by preference bound to a carrier substance that is soluble or dispersible in water. Mixtures of paraffins and bistearylethylenediamide are particularly preferred in this context.

The ingredients to be selected, as well as the conditions under which the agent is used, for example temperature, pH, ionic strength, redox conditions, or mechanical influences, should be optimized for the particular cleaning problem. Usual temperatures for washing and cleaning agents, for example, are in the ranges from 10° C. for manual agents to 40° C. and 60° C. and up to 95° C. for automatic agents or industrial applications. Because the temperature in modern washing machines and dishwashers is usually steplessly adjustable, all intermediate steps of temperature are also included. The ingredients of the relevant agents are, by preference, coordinated with one another. Synergies with regard to cleaning performance are preferred. Particularly preferred in this regard are synergies that are present in a temperature range between 20° C. and 60° C., since the protease contained in the agents according to the present invention is also catalytically active in that temperature range.

In a particularly preferred embodiment, an agent, in particular a washing or cleaning agent, according to the present invention further encompasses
5 wt % to 70 wt %, in particular 5 wt % to 30 wt %, surfactants, and/or
10 wt % to 65 wt %, in particular 12 wt % to 60 wt %, water-soluble or water-dispersible inorganic builder material, and/or
0.5 wt % to 10 wt %, in particular 1 wt % to 8 wt %, water-soluble organic builder substances, and/or
0.01 to 15 wt % solid inorganic and/or inorganic acids or acid salts, and/or
0.01 to 5 wt % complexing agent for heavy metals, and/or
0.01 to 5 wt % anti-gray agent, and/or
0.01 to 5 wt % color transfer inhibitor, and/or
0.01 to 5 wt % foam inhibitor.

Optionally, the agent can further encompass optical brightener, preferably from 0.01 to 5 wt %.

The manufacture of solid agents according to the present invention presents no difficulties and can be accomplished in known fashion, for example by spray-drying or granulation; enzymes and any further thermally sensitive ingredients, such as e.g. bleaching agents, can if applicable be added separately later on. A method comprising an extrusion step is preferred for the manufacture of agents according to the present invention having an elevated bulk weight, in particular in the range from 650 g/l to 950 g/l.

For the manufacture of agents according to the present invention in the form of tablets, which can be single-phase or multiple-phase, single-colored or multi-colored, and in particular can be made up of one layer or of multiple layers, in particular two layers, it is preferable to proceed in such a way that all the constituents (if applicable, of a respective layer) are mixed together in a mixer, and the mixture is compressed by means of conventional tablet presses, for example eccentric presses or rotary presses, at compression pressures in the range from approximately 50 N to 100 kN, by preference at 60 to 70 kN. With multi-layer tablets in particular, it may be advantageous if at least one layer is pre-compressed. This is carried out preferably at compression pressures between 5 and 20 kN, in particular at 10 to 15 kN. Break-resistant tablets that are nevertheless sufficiently rapidly soluble under the utilization conditions, having fracture strength and flexural strength values normally from 100 to 200 N but preferably above 150 N, are thereby obtained without difficulty. A tablet manufactured in this fashion preferably has a weight from 10 to 50 g, in particular from 15 g to 40 g. The tablets can have any three-dimensional shape, for example round, oval, or polygonal, intermediate shapes also being possible. Corners and edges are advantageously rounded. Round tablets by preference have a diameter from 30 mm to 40 mm. In particular, the size of polygonal or cuboidal tablets, which are introduced predominantly via the metering apparatus of, for example, the automatic dishwasher, depends on the geometry and volume of that metering apparatus. Embodiments that are preferred by way of example have a base outline of (20 to 30 mm)×(34 to 40 mm), in particular of 26×36 mm or 24×38 mm.

Liquid or pasty agents according to the present invention in the form of solutions containing usual solvents are generally manufactured by simply mixing the ingredients, which can be introduced into an automatic mixer in substance or as a solution.

Embodiments of the present invention thus encompass all such solid, powdered, liquid, gelled, or pasty administration forms of the agent, which if applicable can also be made up of multiple phases and can be present in compressed or uncompressed form. A further embodiment of the invention is therefore represented by agents which are characterized in that they exist as one-component systems. Such agents are preferably made up of one phase. Agents according to the present invention can, however, of course also be made up of multiple phases. In a further embodiment of the invention, the washing or cleaning agent is therefore characterized in that it is divided into multiple components.

Further included among the solid administration forms according to the present invention are extrudates, granulates, tablets, or pouches, which can be present both in large containers and packaged in portions. Alternatively, the agent exists as a pourable powder, in particular having a bulk weight from 300 g/l to 1200 g/l, in particular 500 g/l to 900 g/l or 600 g/l to 850 g/l.

In addition, agents according to the present invention can also be liquid, gelled, or pasty. A further embodiment of the invention is therefore characterized in that the washing or cleaning agent exists in liquid, gelled, or pasty form, in particular in the form of a nonaqueous liquid washing agent or a nonaqueous paste or in the form of an aqueous liquid washing agent or a hydrous paste.

The washing or cleaning agent according to the present invention can be packaged in a receptacle, by preference an air-permeable receptacle, from which it is released shortly before use or during the washing operation. In particular, the protease contained in the agent and/or further ingredients of the agent can further be encased with a substance that is impermeable to the enzyme at room temperature or in the absence of water, which substance becomes permeable to the enzyme under utilization conditions. One such embodiment of the invention is thus characterized in that the protease is encased with a substance that is impermeable to the protease at room temperature or in the absence of water.

Agents according to the present invention can contain exclusively a protease. Alternatively, they can also contain further proteases or other enzymes, in a concentration useful for the effectiveness of the agent. A further subject of the invention is thus represented by agents that moreover encompass one or more additional enzymes, all enzymes established in the existing art for these purposes being usable in principle. All enzymes that can display catalytic activity in the agent according to the present invention are preferably usable as further enzymes, in particular proteases, amylases, cellulases, hemicellulases, mannanases, tannases, xylanases, xanthanases, β-glucosidases, carrageenases, oxidases, oxidoreductases, or lipases, as well as, by preference, mixtures thereof. These enzymes are in principle of natural origin; proceeding from the natural molecules, improved variants are available for use in washing and cleaning agents and are used in correspondingly preferred fashion. Agents according to the present invention contain enzymes by preference in total quantities from 1×10⁻⁸ to 5 weight percent, based on active protein. The enzymes are contained in agents according to the present invention preferably from 0.001 to 5 wt %, more preferably from 0.01 to 5 wt %, even more preferably from 0.05 to 4 wt %, and particularly preferably from 0.075 to 3.5 wt %, such that each enzyme that is contained can be present in the aforesaid quantitative ratios.

The protein concentration can be determined using known methods, for example the BCA method (bicinchoninic acid; 2,2′-biquinolyl-4,4′-dicarboxylic acid) or the biuret method (A. G. Gornall, C. S. Bardawill, and M. M. David, J. Biol. Chem. 177 (1948), pp. 751-766). Particularly preferably, the further enzymes support the action of the agent, for example the cleaning performance of a washing or cleaning agent, in terms of specific stains or spots. Particularly preferably, the enzymes exhibit synergistic effects in terms of their action with respect to specific stains or spots, i.e. the enzymes contained in the agent composition support one another in terms of their cleaning performance. Synergistic effects can occur not only between different enzymes, but also between one or more enzymes and further ingredients of the agent according to the present invention. In a further preferred embodiment of the invention, the agent according to the present invention is thus characterized in that it contains at least one further enzyme, which is a protease, amylase, cellulase, hemicellulase, mannanase, tannase, xylanase, xanthanase, β-glucosidase, carrageenase, oxidase, oxidoreductase, or a lipase.

The enzymes used in the agents according to the present invention either derive originally from microorganisms, for example the genera Bacillus, Streptomyces, Humicola, or Pseudomonas, and/or are produced by suitable microorganisms in accordance with biotechnological methods known per se, for example by transgenic expression hosts of Bacillus species or by filamentous fungi.

The protease activity in such agents can be ascertained in accordance with the method described in Tenside, Vol. 7 (1970), pp. 125-132. It is indicated accordingly in PU (protease units).

When comparing the performance of two washing-agent enzymes, e.g. in the Examples of the present Application, a distinction must be made between equal-protein and equal-activity utilization. Equal-protein utilization is applied especially in the context of preparations obtained by genetic engineering that are largely free of secondary activity. The reason is that it is thereby possible to ascertain whether the same quantities of protein—as an indication of the fermentative production yield—leads to comparable results. As the respective ratios of active substance to total protein (the values for specific activity) move apart, an equal-activity comparison is recommended, since this compares the respective enzymatic properties. It is generally the case that a low specific activity can be compensated for by adding a larger quantity of protein. This is ultimately an economic judgment.

A separate subject of the invention is represented by the use of an above-described agent according to the present invention for removing protease-sensitive stains on textiles or hard surfaces, i.e. for the cleaning of textiles or hard surfaces.

This is because agents according to the present invention can, especially in accordance with the properties described above, be used to eliminate protein-containing contaminants from textiles or from hard surfaces. Embodiments are represented by, for example, hand laundering, manual removal of spots from textiles or from hard surfaces, or use in connection with an automatic method.

In a preferred embodiment of this use, the relevant agents according to the present invention, by preference washing or cleaning agents, respectively, are made available in accordance with one of the embodiments set forth above.

A further separate subject of the invention is represented by methods for cleaning textiles or hard surfaces in which an agent according to the present invention is used in at least one of the method steps. The method for cleaning textiles or hard surfaces is therefore characterized in that an agent according to the present invention is utilized in at least one method step.

Included thereamong are both manual and automatic methods, automatic methods being preferred because of their more precise controllability with regard, for example, to the quantities and contact times used.

Methods for cleaning textiles are generally notable for the fact that, in multiple method steps, different substances having cleaning activity are applied onto the material to be cleaned and are washed off after the contact time, or that the material to be cleaned is treated in another manner with a washing agent or with a solution of said agent. The same applies to methods for cleaning all materials other than textiles, which are grouped under the term “hard surfaces.” All conceivable washing or cleaning methods can be enhanced, in at least one of the method steps, with an agent according to the present invention, and then represent embodiments of the present invention.

In a further preferred embodiment of such methods, the relevant enzymes according to the present invention are made available in the context of one of the formulations set forth above for agents according to the present invention, by preference washing or cleaning agents, respectively, according to the present invention.

Because preferred enzymes according to the present invention of course already possess a protein-dissolving activity, and display it even in media that otherwise possess no cleaning power, for example in pure buffer, an individual sub-step of a method of this kind for automatic cleaning of textiles can consist in applying an enzyme according to the present invention as the only component having cleaning activity, if desired in addition to stabilizing compounds, salts, or buffer substances. This represents a particularly preferred embodiment of the present invention.

As already explained above, a further object of the present invention is to make available protease enzymes that are suitable for use in agents according to the present invention and are notable for good, preferably for improved proteolytic activity with respect to at least one stain, by preference with respect to multiple stains, in the temperature range recited above, when they are used in agents according to the present invention. This object is achieved by proteases which encompass an amino acid sequence that, in a segment of at least 100 continuously connected amino acids residues, is at least 97.2%, in particular with increasing preference at least 97.25%, 97.5%, 97.75%, 98%, 98.25%, 98.5%, 98.75%, 99%, 99.25%, 99.5%, 99.75%, and very particularly preferably 100%, identical to the amino acid sequence indicated in SEQ ID NO. 2, or is at least 93.7%, in particular with increasing preference at least 94%, 94.25%, 94.5%, 94.75%, 95%, 95.25%, 95.5%, 95.75%, 96%, 96.25%, 96.5%, 96.75%, 97%, 97.25%, 97.5%, 97.75%, 98%, 98.25%, 98.5%, 98.75%, 99%, 99.25%, 99.5%, 99.75%, and very particularly preferably 100%, identical to the amino acid sequence indicated in SEQ ID NO. 6, or is at least 92.4%, in particular with increasing preference at least 92.75%, 93%, 93.25%, 93.5%, 93.75%, 94%, 94.25%, 94.5%, 94.75%, 95%, 95.25%, 95.5%, 95.75%, 96%, 96.25%, 96.5%, 96.75%, 97%, 97.25%, 97.5%, 97.75%, 98%, 98.25%, 98.5%, 98.75%, 99%, 99.25%, 99.5%, 99.75%, and very particularly preferably 100%, identical to the amino acid sequence indicated in SEQ ID NO. 8, or is at least 96.8%, in particular with increasing preference at least 97%, 97.25%, 97.5%, 97.75%, 98%, 98.25%, 98.5%, 98.75%, 99%, 99.25%, 99.5%, 99.75%, and very particularly preferably 100%, identical to the amino acid sequence indicated in SEQ ID NO. 10, or is at least 92.4%, in particular with increasing preference at least 92.75%, 93%, 93.25%, 93.5%, 93.75%, 94%, 94.25%, 94.5%, 94.75%, 95%, 95.25%, 95.5%, 95.75%, 96%, 96.25%, 96.5%, 96.75%, 97%, 97.25%, 97.5%, 97.75%, 98%, 98.25%, 98.5%, 98.75%, 99%, 99.25%, 99.5%, 99.75%, and very particularly preferably 100%, identical to the amino acid sequence indicated in SEQ ID NO. 12, and in particular by one that exists naturally in a bacterial strain of the species Stenotrophomonas maltophilia.

Such proteases therefore represent a further subject of the invention. In further preferred embodiments of the invention, the protease is characterized in that the aforesaid identity indications are referred to a segment of, with increasing preference, 150, 200, 250, 300, 350, 400, 450, 477, 500, 550, or 578 continuously connected amino acid residues. In a preferred embodiment, the segment is selected in such a way that its C-terminus corresponds to position 470 or position 471 or position 472 or position 473 or position 474 or position 475 or position 476 or position 477 or position 478 or position 479 or position 480, with reference to SEQ ID NO. 2. This means that in an alignment, the C-terminal amino acid of the segment according to the present invention is associated with the aforesaid position in SEQ ID NO. 2, wherein the C-terminal amino acid of the segment and the corresponding amino acid in SEQ ID NO. 2 do not need to be identical, but can be identical. What is relevant is the position-related association of the amino acids, not their conformity. Reference has been made above to the meaning “segments” according to the present invention. In a further embodiment of the invention, the protease is characterized in that the aforesaid identity indications are referred to the amino acid sequences indicated in SEQ ID NO. 2 or SEQ ID NO. 6 or SEQ ID NO. 8 or SEQ ID NO. 10 or SEQ ID NO. 12.

These identity indications were ascertained by sequence comparisons between proteases according to the present invention and a protease having the designation StmPr2, which represents the most similar protease with reference respectively to the amino acid sequences in accordance with SEQ ID NO. 2 or SEQ ID NO. 6 or SEQ ID NO. 8 or SEQ ID NO. 10 or SEQ ID NO. 12. The amino acid sequence of this protease StmPr2 is deposited under accession numbers AAP13815 and AY253983 (nucleic acid sequence and corresponding amino acid sequence, respectively) in the publicly accessible database of the NCBI (National Center for Biotechnology Information, 8600 Rockville Pike, Bethesda, Md. 20894), and is appended to this Application in accordance with SEQ ID NO. 4. All the sequence comparisons and determinations of homology values and/or identity values carried out in this patent Application were carried out using the Vector NTI® Suite 7.0 computer program obtainable from InforMax, Inc., Bethesda, USA, using the predefined default parameters. This sequence comparison yielded the following identity values:

| Comparison of | with | % identity |
|---|---|---|
| SEQ ID NO. 2 | StmPr2 (AAP13815/AY253983; | 97.1 |
| | SEQ ID NO. 4 | |
| SEQ ID NO. 6 | StmPr2 (AAP13815/AY253983; | 93.6 |
| | SEQ ID NO .4 | |
| SEQ ID NO. 8 | StmPr2 (AAP13815/AY253983; | 92.3 |
| | SEQ ID NO. 4 | |
| SEQ ID NO. 10 | StmPr2 (AAP13815/AY253983; | 96.7 |
| | SEQ ID NO. 4 | |
| SEQ ID NO. 12 | StmPr2 (AAP13815/AY253983; | 92.3 |
| | SEQ ID NO. 4 | |

All proteases that, as described above, are at least 97.2% identical to SEQ ID NO. 2 or to a segment thereof, at least 93.7% identical to SEQ ID NO. 6 or to a segment thereof, at least 92.4% identical to SEQ ID NO. 8 or to a segment thereof, at least 96.8% identical to SEQ ID NO. 10 or to a segment thereof, and at least 92.4% identical to SEQ ID NO. 12 or to a segment thereof, are therefore included in the range of protection.

In a further preferred embodiment of the invention, the protease is characterized in that its washing performance corresponds at least to that of a protease that encompasses an amino acid sequence that corresponds to the amino acid sequence indicated in SEQ ID NO. 12. or to the amino acid sequence indicated in SEQ ID NO. 12 in a segment of, with increasing preference, 100, 150, 200, 250, 300, 350, 400, 450, 477, 500, 550, 578 continuously connected amino acid residues, the washing performance being determined in a washing system that contains a washing agent at a dosing ratio of between 4.5 and 7.0 grams per liter of washing bath and contains the protease, the proteases to be compared being used in equal-activity fashion and the washing performance being determined with respect to a whole egg/carbon black or whole egg/pigment stain on cotton, in particular the 10N whole egg/pigment on cotton stain, by measuring the degree of whiteness of the washed textiles, the washing operation being performed for at least 30 minutes, optionally 60 minutes, at a temperature of 40° C., and the water having a water hardness of between 15.5 and 16.5° German hardness.

The indications and explanations above with regard to comparison of the washing performance of the proteases apply analogously.

For purposes of the present Application, an “enzyme” is to be understood as a protein that performs a specific biocatalytic function. For purposes of the present Application, a “protease” is understood as an enzyme that catalyzes the hydrolysis of peptide bonds and is thereby capable of cleaving peptides or proteins.

For purposes of the present Application, a “protein” is to be understood as a polymer that is assembled from the natural amino acids, has a largely linear structure, and usually assumes a three-dimensional structure in order to perform its function, i.e. a polypeptide. In the present Application, a “peptide” refers to the naturally occurring proteinogenic L-amino acids designated using the internationally accepted one- and three-letter codes.

Numerous proteins are formed as so-called preproteins, i.e. together with a signal peptide. This is then to be understood as the N-terminal portion of the protein, whose function usually consists in ensuring transfer of the protein, once formed, out of the producing cell into the periplasma or the surrounding medium, and/or correct folding thereof. Under natural conditions, the signal peptide is then cleaved off from the rest of the protein by a signal peptidase, so that the protein exerts its actual catalytic activity without the N-terminal amino acids that were initially present. Proproteins are inactive precursors of proteins. Their parents having a signal sequence are referred to as preproproteins. For industrial applications, the mature peptides (i.e. the enzymes processed after their production) are preferred, because of their enzymatic activity, over the preproteins. The proteins can be modified, after manufacture of the polypeptide chain, by the cells that produce them, for example by the addition of sugar molecules, formylation, amination, etc. Such modifications are referred to as “posttranslational” modifications. These posttranslational modifications can, but do not necessarily, have an influence on the protein's function.

A comparison with known enzymes that are deposited, for example, in generally accessible databases allows the activity of an enzyme in question to be deduced from the amino-acid or nucleotide sequence. This activity can be qualitatively or quantitatively modified by other regions of the protein that do not participate in the actual reaction. This might involve, for example, enzyme stability, activity, reaction conditions, or substrate specificity.

A comparison of this kind is made by mutual association of similar successions in the nucleotide or amino-acid sequences of the proteins in question. This is called “homologation.” A tabular association of the relevant positions is referred to as an “alignment.” When nucleotide sequences are analyzed, consideration must in turn be given to both complementary strands, and to all three possible reading scans in each case, and to the degeneracy of the genetic code and the organism-specific use of the codon (codon usage). Alignments are now prepared using computer programs, for example the FASTA or BLAST algorithms; this procedure is described, for example, by D. J. Lipman and W.R. Pearson (1985) in Science, Vol. 227, pp. 1435-1441.

A combination of all the positions that are in conformity in the sequences that have been compared is referred to as a “consensus sequence.”

A comparison of this kind also allows conclusions as to the similarity or homology of the sequences that are being compared. This is reproduced as a percentage identity, i.e. the proportion of identical nucleotides or amino acid residues at the same positions or in positions corresponding to one another in an alignment. A broader construction of the term “homology” also incorporates the conserved amino acid exchanges into this value. The term used in then “percentage similarity.” Such conclusions can be reached over entire proteins or genes, or only over individual regions.

Homologous regions of different proteins are defined by conformities in the amino acid sequence. These can also be characterized by identical function. It can range up to complete identity in very small regions (so-called “boxes”) that encompass only a few amino acids and usually perform functions essential to overall activity. The “functions” of the homologous regions are to be understood as very small sub-functions of the function performed by the entire protein, for example the formation of individual hydrogen bridge bonds in order to complex a substrate or a transition complex.

In a further preferred embodiment of the invention, the protease according to the present invention is characterized in that it exists naturally in a bacterial strain of the species Stenotrophomonas maltophilia. Alternatively, the protease can also exist naturally in a bacterial strain of the species Stenotrophomonas rhizophilia.

Proteases, and enzymes in general, can be further developed using a variety of methods, e.g. targeted genetic modification by way of mutagenesis methods, and optimized for specific purposes or in terms of special properties, for example catalytic activity, stability, etc.

It is furthermore commonly known from the existing art that advantageous properties of individual mutations, e.g. individual point mutations, can supplement one another. A protease already optimized in terms of specific properties, e.g. in terms of its stability with regard to surfactants or other components, can be additionally further developed according to the present invention.

A further subject of the invention is thus represented by proteases which are characterized in that they are obtainable, from a protease according to the present invention constituting a starting molecule, by fragmentation or by deletion, insertion, or substitution mutagenesis, and encompass an amino acid sequence that is in conformity with the starting molecule over a length of at least 100 and, with increasing preference, at least 150, 200, 250, 300, 350, 400, and very particularly preferably at least 450 continuously connected amino acid positions. Also encompassed are proteases that are obtainable from the aforementioned proteases according to the present invention by derivatization or inversion mutation, it being of course mandatory in this context that the aforementioned continuously connected amino acid positions be maintained.

“Fragments” are understood as all proteins or peptides that are smaller than natural proteins and, for example, can also be obtained synthetically. They can be associated with the relevant complete proteins on the basis of their amino acid sequences. They can, for example, assume similar structures or perform proteolytic activities or sub-activities, for example complexing of a substrate. Fragments and deletion variants of starting proteins are similar in principle, but whereas fragments tend to represent smaller pieces, deletion mutants tend to lack only short regions, and therefore only individual sub-functions.

For purposes of the present Application, “chimeric” or “hybrid” proteins are to be understood, as those proteins whose sequence encompasses the sequences or subsequences of at least two starting proteins. The starting proteins can derive, in this context, from different organisms or the same organism. Chimeric or hybrid proteins can be obtained, for example, by recombination mutagenesis. The purpose of such recombination can be, for example, to bring about or modify a specific enzymatic function with the aid of the fused-on protein portion. It is immaterial for purposes of the present invention whether a chimeric protein of this kind is made up of an individual polypeptide chain or of multiple subunits over which different functions can be distributed.

Proteins obtained by insertion mutation are understood as those variants that have been obtained by inserting a protein fragment into the starting sequences. They may be classified as chimeric proteins because of their similarity in principle. They differ from them only in terms of the relationship between the size of the unmodified protein portion and the size of the protein as a whole. The proportion of foreign protein in such insertion-mutated proteins is smaller than it is in chimeric proteins.

Inversion mutagenesis, i.e. a partial sequence inversion, can be regarded as a special instance of both deletion and insertion. The same applies to a regrouping, deviating from the original amino acid sequence, of different molecule parts. Such a regrouping can be regarded as a deletion variant, an insertion variant, and a shuffling variant of the original protein.

For purposes of the present Application, “derivatives” are understood as those proteins whose pure amino-acid chain has been chemically modified. Such derivatization operations can be performed, for example, biologically by the host cell in conjunction with protein biosynthesis. Molecular-biological methods can be used for this purpose. They can also, however, be carried out chemically, e.g. by chemical conversion of a side chain of an amino acid, or by covalent bonding of a different compound onto the protein. Such a compound can also, for example, involve other proteins, which are bound, for example, via bifunctional chemical compounds to proteins according to the present invention. Modifications of this kind can, for example, influence the substrate specificity or intensity of bonding to the substrate, or can bring about a temporary blockage of enzymatic activity if the coupled-on substance is an inhibitor. This is useful, for example, for the period of storage. “Derivatization” is likewise to be understood as covalent bonding to a macromolecular carrier, and also as a non-covalent inclusion into suitable macromolecular cage structures.

Proteins can also be combined, by reaction with an antiserum or a specific antibody, into groups of immunologically related proteins. The members of a group are notable for the fact that they comprise the same antigenic determinants that are recognized by an antibody.

A further subject of the present invention is therefore constituted by proteases which are characterized in that they comprise at least one, and with increasing preference two, three, or four, antigenic determinants in conformity with a protease according to the present invention.

For purposes of the present invention, all enzymes, proteins, fragments, and derivatives, provided they do not need to be explicitly addressed as such, are grouped together under the general term “proteins.”

The pH profile of the enzymes according to the present invention is compatible with the requisite pH for industrial use, and with typical agents according to the present invention, in particular products such as washing and cleaning agents.

Further subjects of the invention are constituted by nucleic acid molecules that code for a protease according to the present invention, and by vectors containing such a nucleic acid.

In a preferred embodiment of the invention, the nucleic acid that codes for a protease according to the present invention encompasses a nucleic acid sequence that, in a segment of at least 300 continuously connected nucleotides,

is at least 95%, in particular with increasing preference at least 95.5%, 96%, 96.5%, 97%, 97.25%, 97.5%, 97.75%, 98%, 98.25%, 98.5%, 98.75%, 99%, 99.25%, 99.5%, 99.75%, and very particularly preferably 100%, identical to the nucleic acid sequence indicated in SEQ ID NO. 1, or
is at least 90%, in particular with increasing preference at least 90.5%, 91%, 91.5%, 92%, 92.5%, 93%, 93.5%, 94%, 94.5%, 95%, 95.5%, 96%, 96.5%, 97%, 97.25%, 97.5%, 97.75%, 98%, 98.25%, 98.5%, 98.75%, 99%, 99.25%, 99.5%, 99.75%, and very particularly preferably 100%, identical to the nucleic acid sequence indicated in SEQ ID NO. 5, or
is at least 90%, in particular with increasing preference at least 90.5%, 91%, 91.5%, 92%, 92.5%, 93%, 93.5%, 94%, 94.5%, 95%, 95.5%, 96%, 96.5%, 97%, 97.25%, 97.5%, 97.75%, 98%, 98.25%, 98.5%, 98.75%, 99%, 99.25%, 99.5%, 99.75%, and very particularly preferably 100%, identical to the nucleic acid sequence indicated in SEQ ID NO. 7, or
is at least 90%, in particular with increasing preference at least 90.5%, 91%, 91.5%, 92%, 92.5%, 93%, 93.5%, 94%, 94.5%, 95%, 95.5%, 96%, 96.5%, 97%, 97.25%, 97.5%, 97.75%, 98%, 98.25%, 98.5%, 98.75%, 99%, 99.25%, 99.5%, 99.75%, and very particularly preferably 100%, identical to the nucleic acid sequence indicated in SEQ ID NO. 9, or
is at least 90%, in particular with increasing preference at least 90.5%, 91%, 91.5%, 92%, 92.5%, 93%, 93.5%, 94%, 94.5%, 95%, 95.5%, 96%, 96.5%, 97%, 97.25%, 97.5%, 97.75%, 98%, 98.25%, 98.5%, 98.75%, 99%, 99.25%, 99.5%, 99.75%, and very particularly preferably 100%, identical to the nucleic acid sequence indicated in SEQ ID NO. 11, or
is at least 90%, in particular with increasing preference at least 90.5%, 91%, 91.5%, 92%, 92.5%, 93%, 93.5%, 94%, 94.5%, 95%, 95.5%, 96%, 96.5%, 97%, 97.25%, 97.5%, 97.75%, 98%, 98.25%, 98.5%, 98.75%, 99%, 99.25%, 99.5%, 99.75%, and very particularly preferably 100%, identical to the nucleic acid sequence indicated in SEQ ID NO. 13.

Particularly preferably, a nucleic acid of this kind exists naturally in a bacterial strain of the species Stenotrophomonas maltophilia.

In further preferred embodiments of the invention, the nucleic acid is characterized in that the identity indications above are referred to a segment of, with increasing preference, 450, 600, 750, 900, 1050, 1200, 1350, 1431, 1500, 1650, 1734 continuously connected nucleic acids. In further preferred fashion, a segment of this kind contains no stop codon. In a further preferred embodiment, the segment is selected in such a way that the last codon coding for an amino acid (base triplet) codes for a C-terminal amino acid of the segment, said amino acid corresponding to 470 or position 471 or position 472 or position 473 or position 474 or position 475 or position 476 or position 477 or position 478 or position 479 or position 480, with reference to SEQ ID NO. 2. This means that in an alignment, the C-terminal amino acid of the segment according to the present invention is associated with the aforesaid position in SEQ ID NO. 2, wherein the C-terminal amino acid of the segment and the corresponding amino acid in SEQ ID NO. 2 do not need to be identical, but can be identical. What is relevant is the position-related association of the amino acids, not their conformity. In further particular embodiments of the invention, the protease is characterized in that the aforesaid identity indications are referred to the nucleic acid sequences indicated in SEQ ID NO. 1 or SEQ ID NO. 5 or SEQ ID NO. 7 or SEQ ID NO. 9 or SEQ ID NO. 11 or SEQ ID NO. 13.

The nucleic acids according to SEQ ID NO. 11 and SEQ ID NO. 13 differ in only a few base pairs, namely two base pairs, even though they derive from different Stenotrophomonas maltophilia strains. They therefore code for amino acid sequences that are identical to one another, which are indicated in SEQ ID NO. 12 and SEQ ID NO. 14.

For purposes of the present Application, “nucleic acids” are to be understood as the molecules, constructed naturally from nucleotides and serving as information carriers, that code for the linear amino-acid sequence in proteins or enzymes. They can be present as a single strand, as a single strand complementary to the latter single strand, or as a double strand. The nucleic acid DNA, constituting the naturally more durable information carrier, is preferred for molecular-biological work. For implementation of the invention in a natural environment, however, for example in an expressing cell, an RNA is formed, and RNA molecules that are essential to the invention therefore also represent embodiments of the present invention.

In DNA, the sequences of both complementary strands are to be taken into account in all three possible reading patterns in each case. Consideration is also to be given to the fact that different codon triplets can code for the same amino acids, so that a specific amino-acid succession can be derived from multiple different nucleotide sequences that possibly exhibit little identity; this is referred to as “degeneracy” of the genetic code. In addition, various organisms exhibit differences in how these codons are used. For these reasons, both amino-acid sequences and nucleotide sequences must be included in the consideration of the range of protection. All nucleotide sequences that can code one of the proteases described above are therefore also included in the invention. One skilled in the art is capable of determining these nucleotide sequences unequivocally, since despite the degeneracy of the genetic code, individual codons are to be associated with defined amino acids. One skilled in the art can therefore, proceeding from a are to be regarded in each case as only one exemplifying coding for a specific amino acid sequence.

The information unit corresponding to a protein is also referred to, for purposes of the present Application, as a “gene”.

The present invention encompasses the production of recombinant proteins. These are to be understood according to the present invention as all genetic-engineering or microbiological methods based on the fact that the genes for the proteins of interest are introduced into a host cell suitable for production, and are transcribed and translated thereby. It is appropriate for the relevant genes to be transferred in via vectors, in particular expression vectors, but also via those which cause the gene of interest in the host cell to be inserted into a genetic element (such as the chromosome or other vectors) that is already present. The functional element made up of gene and promoter and possible further genetic elements is referred to according to the present invention as an “expression cassette.” For this, however, it need not necessarily also exist as a physical unit.

It is possible for one skilled in the art, using methods commonly known today such as, for example, chemical synthesis or polymerase chain reaction (PCR) in combination with standard molecular-biological and/or protein-chemical methods, to manufacture the corresponding nucleic acids, to the point of complete genes, on the basis of known DNA and/or amino acid sequences. Such methods are known, for example, from Sambrook, J., Fritsch, E. F., and Maniatis, T, 2001, Molecular cloning: a laboratory manual, 3rd edition, Cold Spring Laboratory Press.

Modifications to the nucleotide sequence, such as those that can be brought about, for example, using molecular-biological methods known per se, are referred to as “mutations.” Known types are, for example (depending on the nature of the change), deletion, insertion, or substitution mutations, or those in which different genes or portions of genes are fused or recombined with one another, which latter are gene mutations. The associated organisms are referred to as “mutants.” The proteins derived from mutated nucleic acids are referred to as “variants.” For example, fusions and deletion, insertion, or substitution mutations result in fusion genes or deletion-, insertion-, or substitution-mutated genes and, on the protein level, in corresponding fusion proteins or deletion, insertion, or substitution variants, respectively.

For purposes of the present invention, “vectors” are understood as elements, made up of nucleic acids, that contain a gene of interest as a characterizing nucleic-acid region. They enable the gene to be established as a stable genetic element in a species or a cell line over multiple generations or cell divisions. Vectors are, in particular when used in bacteria, especially plasmids, i.e. circular genetic elements. A distinction is made in genetic engineering on the one hand between those vectors (so-called “cloning vectors”) that serve for storage and thus also to a certain extent for genetic-engineering work, and on the other hand those that perform the function of implementing the gene of interest in the host cell, i.e. enabling expression of the relevant protein. These vectors are referred to as “expression vectors.”

In the context of the present invention, the nucleic acid is appropriately cloned into a vector. The molecular-biological dimension of the invention thus consists in vectors having the genes for the corresponding proteins. These can include, for example, those that are derived from bacterial plasmids, from viruses, or from bacteriophages, or predominantly synthetic vectors or plasmids having elements of a wide variety of origins. With the further genetic elements that are respectively present, vectors are capable of establishing themselves in the relevant host cells as stable units over multiple generations. It is immaterial in the context of the present invention whether they establish themselves extrachromosomally as independent units or are integrated into a chromosome. The individual case determines which of the many systems known from the existing art is selected. The critical factor may be, for example, the number of copies that can be achieved; the available selection systems, including principally antibiotic resistances; or culturability of the host cells capable of receiving the vectors.

The vectors constitute suitable starting points for molecular-biological and biochemical investigations of the relevant gene or associated protein, and for further developments according to the present invention, and ultimately for the amplification and production of proteins according to the present invention. They represent embodiments of the present invention insofar as the sequences of the nucleic acid regions according to the present invention that are obtained are respectively located within the homology regions characterized in further detail above.

A further subject of the invention is thus represented by vectors that contain at least one nucleic acid molecule that codes for a protease according to the present invention, in particular a nucleic acid molecule as described above. In a further embodiment of the invention, the vector is characterized in that the vector is a cloning vector. These cloning vectors are suitable, in addition to storage, biological amplification, or selection of the gene of interest, for characterization of the relevant gene, for example via preparation of a restriction map or sequencing. Cloning vectors are therefore also preferred embodiments of the present invention because they represent a transportable and storage-capable form of the claimed DNA. They are also preferred starting points for molecular-biological techniques that are not confined to cells, for example the polymerase chain reaction.

In a further embodiment of the invention, the vector is characterized in that the vector is an expression vector. Expression vectors are chemically similar to cloning vectors, but differ in terms of those subsequences which make them capable of replicating in the host cells or host organisms optimized for the production of proteins, and causing the contained gene to be expressed there. Preferred embodiments are expression vectors that themselves carry the genetic elements necessary for expression. Expression is influenced, for example, by promoters that regulate transcription of the gene. Expression can be accomplished, for example, by the natural promoter originally located in front of that gene, but also (after genetic-engineering fusion) both by a host-cell promoter made available on the expression vector and by a modified or entirely different promoter of another organism or another host cell.

Preferred embodiments are those expression vectors that can be regulated by way of modifications to culture conditions or by the addition of specific compounds, for example cell density or specific factors. Expression vectors allow the associated protein to be produced heterologously, i.e. in a cell or host cell other than the one from which it can be recovered naturally. The cells can by all means belong to different organisms or derive from different organisms. Homologous protein recovery, from a host cell that naturally expresses the gene via a suitable vector, is also within the range of protection of the present invention. This can have the advantage that natural modification reactions that are correlated with translation can be carried out on the resulting protein in exactly the same way in which they would also proceed naturally.

A further embodiment is represented by expression systems in which additional genes, for example those made available on other vectors, influence the production of proteins according to the present invention. These can involve modified gene products or those that are intended to be purified together with the protein according to the present invention, for example in order to influence its enzymatic function. This can involve, for example, other proteins or enzymes, or inhibitors, or those elements that influence interaction with various substrates.

Alternative embodiments of the present invention can also be cell-free expression systems in which protein biosynthesis is accomplished in vitro. Such expression systems are likewise established in the existing art.

A further subject of the invention is a non-human host cell that contains a protease according to the present invention or a fragment thereof, or that can be stimulated to the production thereof, by preference with the use of an expression vector. In vivo synthesis of an enzyme according to the present invention, i.e. synthesis by living cells, requires transfer of the relevant gene into a host cell (so-called “transformation” thereof). All cells are in principle suitable as host cells, i.e. prokaryotic or eukaryotic cells. Those host cells that can be manipulated in genetically advantageous fashion, e.g. as regards transformation using the expression vector and stable establishment thereof, are preferred, for example single-celled fungi or bacteria. In addition, preferred host cells are notable for being readily manipulated in microbiological and biotechnological terms. This refers, for example, to easy culturability, high growth rates, low demands in terms of fermentation media, and good production and secretion rates for foreign proteins. It is often necessary to ascertain experimentally, from the plethora of different systems available in accordance with the existing art, the optimum expression systems for an individual case. Each protein according to the present invention can be recovered in this fashion from a plurality of host cells. Those host cells characterized in that they were obtained after transformation with one of the vectors described above are also preferred. This can refer, for example, to cloning vectors that were introduced for storage and/or modification purposes, for example, into any bacterial strain or into another host cell according to the present invention. Such steps are widespread in the storage and further development of relevant genetic elements. Because the relevant genetic elements can be transferred directly out of these host cells into subsequent host cells suitable for expression, the preceding transformation products are also implementations of the relevant subject of the invention.

Preferred embodiments are represented by those host cells whose activity can be regulated on the basis of genetic regulation elements that are made available, for example, on the expression vector but can also be present a priori in those cells. They can be stimulated to expression, for example, by controlled addition of chemical compounds that serve as activators, by modifying the culture conditions, or when a specific cell density is reached. This makes possible very economical production of the proteins of interest.

Preferred host cells are prokaryotic or bacterial cells. Bacteria are generally notable, as compared with eukaryotes, for shorter generation times and lesser demands in terms of culturing conditions. As a result, economical conditions for recovering proteins according to the present invention can be established. In Gram-negative bacteria, for example Escherichia coli (E. coli), a plurality of proteins are secreted into the periplasmic space, i.e. into the compartment between the two membranes enclosing the cell. This can be advantageous for specific applications. Gram-positive bacteria, on the other hand, such as e.g. bacilli or actinomycetes, or other representatives of the actinomycetals, possess no external membrane, so that secreted proteins are discharged immediately into the nutrient medium surrounding the cells, from which medium, in accordance with a further preferred embodiment, the expressed proteins according to the present invention can be purified directly.

By preference, the host cell is therefore characterized in that it secretes the protein according to the present invention, or a fragment or derivative thereof, into the surrounding medium. A further subject of the invention is thus represented by host cells which are characterized in that they secrete the protease, or a fragment thereof, into the medium surrounding the host cell.

In a further preferred embodiment, the host cell according to the present invention is characterized in that it is a bacterium, in particular one that is selected from the group of the genera Escherichia, Bacillus and Arthrobacter, Streptomyces, Stenotrophomonas and Pseudomonas. Particularly preferably, the host cell is a bacterium that is selected from the group of Escherichia coli, Bacillus lichenifonnis, Bacillus amyloliquefaciens, Bacillus subtilis, Bacillus alcalophilus and Arthrobacter oxidans, Streptomyces lividans, Streptomyces coelicolor, and Stenotrophomonas maltophilia.

The host cells can be modified in terms of their requirements regarding culture conditions, can comprise other or additional selection markers, or can express other or additional proteins. The host cells can, in particular, be ones that, in addition to the protein manufactured according to the present invention, also express further proteins, especially ones of economic interest.

The host cell can, however, also be a eukaryotic cell which is characterized in that it possesses a cell nucleus. A further subject of the invention is therefore represented by a host cell which is characterized in that it possesses a cell nucleus.

In contrast to prokaryotic cells, eukaryotic cells are capable of posttranslationally modifying the protein that is formed. Examples thereof are fungi such as Actinomycetes, or yeasts such as Saccharomyces or Kluyveromyces. This may be particularly advantageous, for example, when the proteins, in connection with their synthesis, are intended to experience specific modifications made possible by such systems. These include, for example, the bonding of low-molecular-weight compounds such as membrane anchors or oligosaccharides.

The host cells according to the present invention are cultured and fermented in a manner known per se, for example in discontinuous or continuous systems. In the former case a suitable nutrient medium is inoculated with the host cells and the product is harvested from the medium after a period of time to be ascertained experimentally. Continuous fermentations are notable for the achievement of a flow equilibrium in which, over a comparatively long period of time, cells die off in part but are also in part renewed, and product can simultaneously be removed from the medium.

Fermentation methods are known from the existing art and represent the actual industrial-scale production step, generally followed by a suitable method for purifying the product that was manufactured, for example the recombinant protein. All fermentation methods that are based on one of the methods set forth above for manufacturing recombinant proteins correspondingly represent preferred embodiments of this subject of the invention.

Optimum conditions for the manufacturing methods that are used, for the host cells, and/or for the proteins to be manufactured, based on the previously optimized culture conditions of the relevant strain according to the knowledge of one skilled in the art, for example in terms of fermentation volume, media composition, oxygen supply, or shaker speed, must be ascertained experimentally in this context.

Fermentation methods which are characterized in that fermentation is carried out by way of an inflow strategy are likewise possible. In this context the media constituents that are consumed during continuous culturing are fed in (the term “feed strategy” is also used). Considerable increases both in cell density and in dry biomass, and/or principally in the activity of the protein of interest, can thereby be achieved.

Analogously to this, the fermentation operation can also be configured so that undesired metabolic products are filtered out, or are neutralized by the addition of a buffer or respectively suitable counterions.

The protein that is manufactured can be harvested subsequently from the fermentation medium. This fermentation method is preferred over product preparation from the dry mass, but requires that suitable secretion markers and transport systems be made available. Without secretion, in some cases purification of the protein from the cell mass is necessary; various methods are also known for this, such as precipitation using e.g. ammonium sulfate or ethanol, or chromatographic purification, if necessary to the point of homogeneity. Most of the industrial methods described, however, should be able to manage with an enriched, stabilized preparation.

All the elements already set forth above can be combined into methods for manufacturing proteins according to the present invention. A plurality of possible combinations of method steps is conceivable for each protein according to the present invention. The optimum method must be ascertained experimentally for each specific individual case.

The proteases according to the present invention can be made available by expression or cloning in the quantity necessary for industrial use.

An independent subject of the invention is therefore also represented by methods for manufacturing a protease according to the present invention.

This includes any method that is suitable for the manufacture of a protease as described above according to the present invention, or that enables a protease according to the present invention to be obtained. These also include, for example, chemical synthesis methods.

Preferred in contrast thereto, however, are all molecular-biological, microbiological, or biotechnological manufacturing methods, established in the existing art and already addressed above in individual aspects, that build on the proteins or nucleic acids according to the present invention and characterized above. In accordance with the above statements, for example, the nucleic acid indicated in the sequence protocol under SEQ ID NO. 1 or SEQ ID NO. 5 or SEQ ID NO. 7 or SEQ ID NO. 9 or SEQ ID NO. 11 or SEQ ID NO. 13, or mutants or sub-sequences obtained therefrom, may be correspondingly drawn upon.

The methods in this context are preferably ones carried out with the use of a previously mentioned nucleic acid, by preference with the use of a vector described above and particularly preferably, by preference, with the use of a previously mentioned host cell that advantageously has been modified by genetic engineering. The reason is that in this fashion, the correspondingly preferred genetic information is made available in microbiologically exploitable form.

Embodiments of the present invention can also, on the basis of the associated nucleic acid sequences, be cell-free expression systems in which protein biosynthesis can be performed in vitro. All the elements already set forth above can also be combined into new methods for manufacturing proteins according to the present invention. A plurality of possible combinations of method steps is conceivable for each protein according to the present invention, so that optimum methods for each specific individual case must be ascertained experimentally.

In accordance with the above statements, among the aforesaid methods those in which the nucleotide sequence has been adapted in one, by preference in multiple codons to the codon usage of the host strain, are further preferred.

A further subject of the invention is represented by methods for cleaning textiles or hard surfaces, in which methods a protease according to the present invention is used in at least one of the method steps. The method for cleaning textiles or hard surfaces is therefore characterized in that a protease according to the present invention is proteolytically active in at least one method step.

Included thereamong are both manual and automatic methods, automatic methods being preferred because of their more precise controllability with regard, for example, to the quantities and contact times used.

Methods for cleaning textiles are generally notable for the fact that, in multiple method steps, various substances having cleaning activity are applied onto the material to be cleaned and are washed off after the contact time, or that the material to be cleaned is treated in another manner with a washing agent or with a solution of said agent. The same applies to methods for cleaning all materials other than textiles, which are grouped under the term “hard surfaces.” All conceivable washing or cleaning methods can be enhanced, in at least one of the method steps, with a protease according to the present invention, and then represent embodiments of the present invention.

In the methods according to the present invention, the protease is used in a quantity from 40 μg to 4 g, by preference from 50 μg to 3 g, particularly preferably from 100 μg to 2 g, and very particularly preferably from 200 μg to 1 g, per application.

In a further preferred embodiment of such methods, the relevant polypeptides according to the present invention are made available in the context of one of the formulations set forth above for agents according to the present invention, by preference washing or cleaning agents according to the present invention.

Embodiments of this subject of the invention are represented by methods for treating textile raw materials or for textile care, in which methods a protease according to the present invention becomes active in at least one of the method steps.

Preferred thereamong are methods for textile raw materials, fibers, or textiles having natural constituents, and very particular for those having wool or silk.

These can be, for example, methods in which materials for processing into textiles are prepared, for example for anti-felting finishing; or, for example, methods that supplement the cleaning of previously worn textiles with a care-providing component. Because of the above-described action of proteases on natural, protein-containing raw materials, preferred embodiments refer to methods for the treatment of textile raw materials, fibers, or textiles having natural constituents, in particular having wool or silk.

A further separate subject of the invention is represented by utilization of an above-described protease according to the present invention for removing protease-sensitive stains on textiles or hard surfaces, i.e. for the cleaning of textiles or hard surfaces.

This is because proteases according to the present invention, especially as a result of the properties described above, can be used to eliminate protein-containing contaminants from textiles or from hard surfaces. Embodiments are represented by, for example, hand laundering, manual removal of spots from textiles or from hard surfaces, or use in connection with an automatic method. In a preferred embodiment of this use, the relevant proteases according to the present invention are made available in accordance with one of the formulations set forth above.

A use according to the present invention of a protease according to the present invention is preferably notable for the fact that the protease is used in a quantity from 40 μg to 4 g, by preference from 50 μg to 3 g, particularly preferably from 100 μg to 2 g, and very particularly preferably from 200 μg to 1 g, per application.

The following Examples explain the invention without, however, limiting it thereto:

All the molecular-biological working steps follow standard methods such as those indicated, for example, in the manual of Fritsch, Sambrook, and Maniatis “Molecular cloning: a laboratory manual,” Cold Spring Harbor Laboratory Press, New York, 1989, or comparable relevant works. Enzymes, kits, and apparatus were used in accordance with the respective manufacturer's instructions.

### EXAMPLE 1

### Identification of Proteolytically Active Stenotrophomonas Maltophilia Strains

The bacterial cultures were wiped onto NB plates (agar nutrient broth; all indications in grams per liter: peptone from casein 3.5; peptone from meat 2.5; peptone from gelatin 2.5; yeast extract 2.5; sodium chloride 5.0; agar 12), and after two to three days wiped onto skim-milk plates (all indications in grams per liter: peptone from casein 5; yeast extract 2.5; glucose 1.0; also: 100 ml/l 10% skim milk solution; 15% agar). After 48 hours of incubation at 25 to 30° C., the cultures having definite lysis haloes were once again inoculated onto NB plates. From these plates, 25 ml liquid culture was then inoculated and incubated for two days at 25 to 30° C.

After cell harvesting, the media supernatant was investigated for protease activity and protease activity was determined. For this, 250 μl azocasein solution (2% azocasein in 50 mM phosphate buffer, pH 7) was incubated with 150 μl enzyme at 37° C. for exactly 30 minutes, 1.2 ml 10% trichloroacetic acid was added to the reaction mixture, and after 15 minutes of incubation the precipitate was centrifuged off at 8000 G. 600 μl supernatant was mixed with 700 μl 1M NaOH, and measured photometrically at a wavelength of 440 nm. 1 unit is the quantity of enzyme necessary to achieve a change in absorption of 1.0 per minute in a cuvette 1 cm thick.

### EXAMPLE 2

### Recombinant Expression of Cloned Proteases According to the Present Invention from Stenotrophomonas maltophilia

The genes coding for proteases according to the present invention were obtained by polymerase chain reaction (PCR) amplification from the genomic DNA of the corresponding strains, in a manner usual in the art. The genes obtained were cloned in suitable expression vectors, the latter were transformed in E. coli BL21-Gold bacteria, and the proteases were expressed recombinantly in those bacteria.

The following proteases according to the present invention from Stenotrophomonas maltophilia were expressed recombinantly, the nomenclature and numbering corresponding to that of the next Example which documents washing performance:

SEQ ID NO. 2 (protease SmP2)
SEQ ID NO. 6 (protease no. 1 in next Example)
SEQ ID NO. 8 (protease no. 5 in next Example)
SEQ ID NO. 10 (protease no. 7 in next Example)
SEQ ID NO. 12 (protease no. 10 in next Example)
SEQ ID NO. 14 (protease no. 6 in next Example)

The clones of the strains were cultivated in shaker culture: 30 ml LM/ampicillin (conc.: 100 μg/ml) medium were inoculated, and shaken overnight at 30° C. and 160 rpm. 200 ml LB/ampicillin medium were inoculated with the overnight culture to an optical density (OD) 600=0.1, and shaken at 30° C. and 200 rpm to an OD 600=0.6 to 0.8. At this point in time, induction of the culture with 0.05 mM IPTG at 20° C. was performed. Harvesting occurred after an induction period of 25 hours.

### EXAMPLE 3

### Ascertaining Washing Performance when Used in Commercially Usual Washing-Agent Formulations

Standardized stained textiles that had been procured from the Eidgenössische Material-Prüfungs- und -Versuchsanstalt [Swiss federal materials testing and research agency], St. Gallen, Switzerland (EMPA), or from wfk Testgewebe GmbH (Christenfeld 10, D-41379 Brüggen-Bracht, Germany) were used for this example. The following stains and textiles were utilized: A (grass on cotton, EMPA 164), B (whole egg/carbon black on cotton, 10N), C (blood/milk on cotton, C-5 (044)).

Using this test material, a variety of washing-agent formulations that differed in terms of the respective protease contained therein were investigated with regard to their washing performance. For this, the preparations were washed for 60 minutes at a temperature of 40° C. The further experimental protocol, in particular the washing-agent composition and dosing ratio, corresponded to the washing system described above and to the procedure described above for ascertaining the washing performance of the proteases according to the present invention. Washing was performed using tap water having a hardness of approximately 16° German hardness.

For the various series of experiments, various proteases according to the present invention from Stenotrophomonas maltophilia strains were added in equal-activity fashion to the washing-agent formulation. The proteases either were expressed recombinantly or were obtained from the culture medium (or as a culture supernatant) of corresponding Stenotrophomonas maltophilia strains, in a manner usual in the art. The protease disclosed in FIG. 2 and SEQ ID NO. 3 of International Application WO 03/057713 was used as a reference enzyme. In the formulations used, this protease exhibited the best washing performance among the subtilisins tested, and therefore represents a suitable reference enzyme (hereinafter referred to as “Reference 1”). In a powdered agent composition, alternatively either a stabilized (and therefore improved-performance) protease variant (BLAP X) of the alkaline protease from Bacillus lentus (Bacillus lentus DSM 5843), which in addition to further protease variants is described in International Application WO 92/21760 (hereinafter referred to as “Reference 2”), or the protease from Bacillus lentus variant F49 (WO 95/23221, hereinafter referred to as “Reference 3”), served as a reference enzyme.

After washing, the degree of whiteness of the washed textiles was measured. Measurement was performed on a Minolta CM508d spectrometer, illuminant D65, in a manner usual in the art. The unit is first calibrated using a white standard supplied therewith. The results obtained are the differential remission units between a washing operation using a washing agent containing a protease, and a control washing operation, carried out in parallel, using a washing agent without protease. The results are summarized in Tables 1 to 3. They allow immediate conclusions to be drawn as the contribution made by the respectively contained enzyme to the washing performance of the agent used.

Table 1 shows the washing results for a liquid agent composition according to the present invention that contains a protease according to the present invention (SmP2, SEQ ID NO. 2), as compared with an agent composition that contains Reference 1 protease. It is evident that the agent composition according to the present invention, and the protease according to the present invention, surpass the subtilisin washing performance at 40° C. on the stains, in particular for stains B (whole egg/carbon black) and C (blood/milk).

TABLE 1

| Stain | SmP2 | Reference 1 |
|---|---|---|
| A | 4.3 | 3.9 |
| B | 10.5 | 3.7 |
| C | 19.6 | 14.7 |

Table 2 shows the washing results for a powdered agent composition according to the present invention that contains a protease according to the present invention (SmP2, SEQ ID NO. 2), as compared with an agent composition that contains Reference 2 protease. It is evident that the agent composition according to the present invention, and the protease according to the present invention, surpass the subtilisin washing performance at 40° C.

TABLE 2

| Stain | SmP2 | Reference 2 |
|---|---|---|
| B | 9.0 | 6.8 |

Tables 3a) to e) show the washing results for further liquid agent compositions according to the present invention that contain proteases from Stenotrophomonas maltophilia (proteases nos. 1 to 8, 10 to 20, and 22 to 25), as compared with an agent composition that contains Reference 1 protease. The amino acid sequences of the proteases numbered 1, 5, 6, 7, and 10 are indicated in SEQ ID NO. 6 (protease no. 1), SEQ ID NO. 8 (protease no. 5), SEQ ID NO. 14 (protease no. 6), SEQ ID NO. 10 (protease no. 7), and SEQ ID NO. 12 (protease no. 10), and the associated nucleic acid sequences in SEQ ID NO. 5, SEQ ID NO. 7, SEQ ID NO. 13, SEQ ID NO. 9, and SEQ ID NO. 11. It is apparent that the agent compositions according to the present invention, and the proteases according to the present invention, surpass the subtilisin washing performance on the stains at 40° C., in particular on stains B (whole egg/carbon black) and C (blood/milk).

TABLE 3
a)

| | Protease no. | | | | | Refer- |
|---|---|---|---|---|---|---|
| Stain | 1 | 2 | 3 | 4 | 5 | ence 1 |
| A | 4.2 | 3.5 | 4.0 | 3.8 | 3.8 | 2.9 |
| B | 9.5 | 8.3 | 8.6 | 8.5 | 9.0 | 2.4 |
| C | 16.5 | 15.5 | 16.2 | 17.0 | 17.1 | 13.8 |

b)

| | Protease no. | | | Refer- |
|---|---|---|---|---|
| Stain | 6 | 7 | 8 | ence 1 |
| A | 4.5 | 4.8 | 3.7 | 1.9 |
| B | 6.5 | 6.7 | 6.6 | 2.4 |
| C | 20.5 | 20.8 | 18.8 | 16.4 |

c)

| | Protease no. | | | Refer- |
|---|---|---|---|---|
| Stain | 10 | 11 | 12 | ence 1 |
| A | 2.0 | 2.5 | 2.5 | 2.1 |
| B | 5.7 | 5.6 | 5.6 | 3.2 |
| C | 17.3 | 15.5 | 16.0 | 15.5 |

d)

| | Protease no. | | | | | | | | Refer- |
|---|---|---|---|---|---|---|---|---|---|
| Stain | 13 | 14 | 15 | 16 | 17 | 18 | 19 | 20 | ence 1 |
| A | 6.4 | 6.0 | 6.0 | 5.8 | 5.6 | 5.6 | 5.4 | 5.8 | 3.5 |
| B | 9.7 | 8.5 | 9.9 | 9.2 | 9.3 | 9.9 | 10.6 | 9.3 | 1.9 |
| C | 22.4 | 21.5 | 22.7 | 19.0 | 22.6 | 23.6 | 24.8 | 22.2 | 14.0 |

e)

| | |
|---|---|
| Protease no. | Refer- |

| Stain | 22 | 23 | 24 | 25 | ence 1 |
|---|---|---|---|---|---|
| A | 2.7 | 1.9 | 2.6 | 3.1 | 1.6 |
| B | 10.2 | 10.1 | 9.9 | 10.4 | 0.0 |
| C | 19.5 | 20.0 | 22.5 | 21.2 | 14.4 |

Table 4 shows the washing results for further powdered agent compositions according to the present invention that contain proteases from Stenotrophomonas maltophilia as indicated, as compared with an agent composition that contains the Reference 3 protease. The amino acid sequences of the proteases numbered 1, 5, 6, 7, and 10 are indicated in SEQ ID NO. 6 (protease no. 1), SEQ ID NO. 8 (protease no. 5), SEQ ID NO. 14 (protease no. 6), SEQ ID NO. 10 (protease no. 7), and SEQ ID NO. 12 (protease no. 10), and the associated nucleic acid sequences in SEQ ID NO. 5, SEQ ID NO. 7, SEQ ID NO. 13, SEQ ID NO. 9, and SEQ ID NO. 11. It is apparent that the agent compositions according to the present invention, and the proteases according to the present invention, surpass the subtilisin washing performance on the stains at 40° C., in particular on stains B (whole egg/carbon black) and C (blood/milk).

TABLE 4

| | Protease no. | | | | | Refer- |
|---|---|---|---|---|---|---|
| Stain | 5 | 10 | 6 | 1 | 7 | ence 1 |
| A | 2.4 | 2.5 | 2.7 | 3.3 | 2.7 | 1.4 |
| B | 7.6 | 7.6 | 5.9 | 7.5 | 7.7 | 7.2 |
| C | 9.2 | 9.0 | 9.3 | 10.0 | 8.7 | 7.3 |

Comparable washing performance values were obtained for liquid and powdered washing agents that contained, instead of the full-length proteases, C-terminal shortened variants of those proteases whose C-terminus corresponded to position 477, associated in an alignment and referred to SEQ ID NO. 2.

### DESCRIPTION OF THE FIGURES

FIG. 1: Sequence comparison (alignment) of the following amino acid sequences:

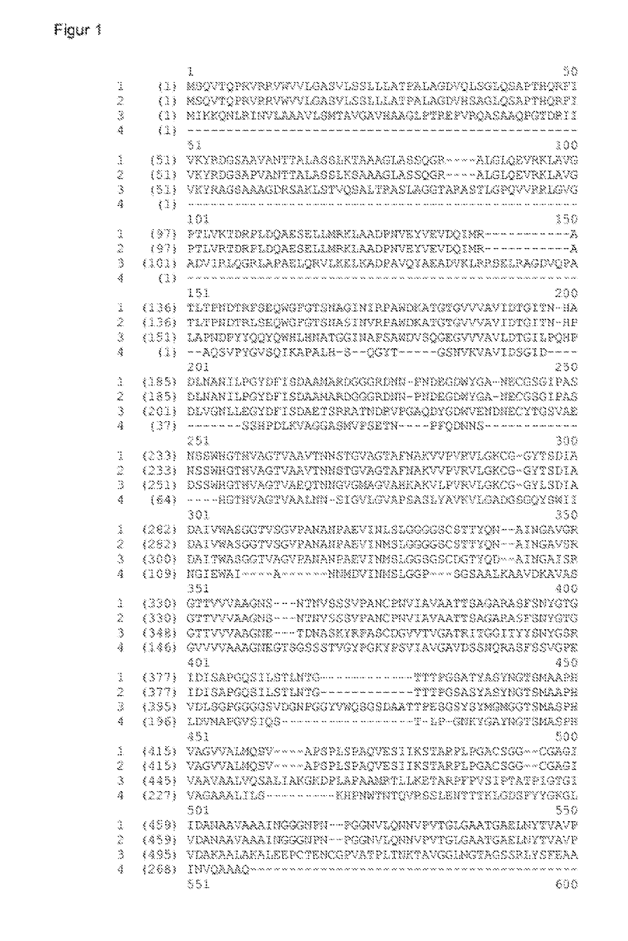

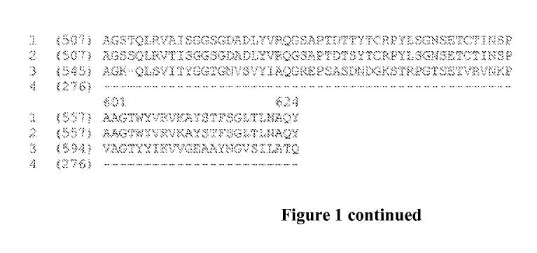

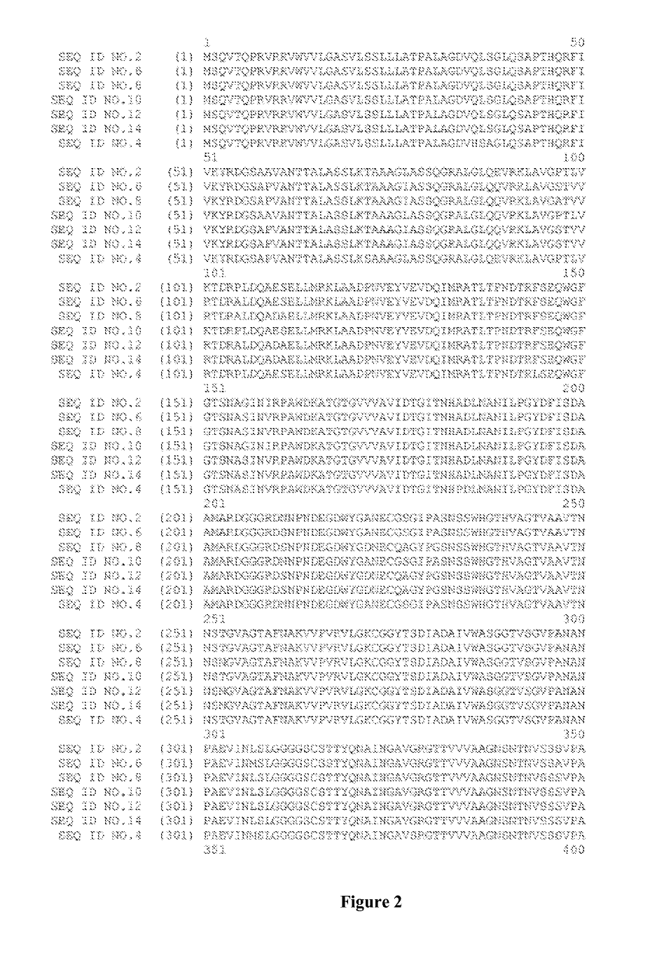

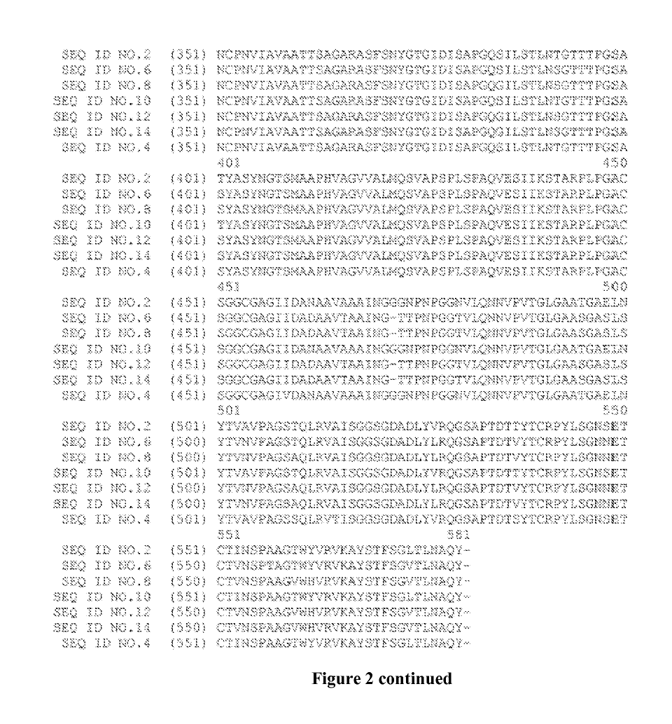

Protease according to the present invention (SmP2) from Stenotrophomonas maltophilia (S. maltophilia DSM 50170) according to SEQ ID NO. 2,

(2) Protease StmPr2 (SEQ ID NO. 4) from Stenotrophomonas maltophilia in accordance with accession nos. AAP13815 and AY253983 (nucleic acid sequence and corresponding amino acid sequence) of the publicly accessible database of the NCBI (National Center for Biotechnology Information, 8600 Rockville Pike, Bethesda, Md. 20894),

(3) Protease StmPr1 (SEQ ID NO. 3) from Stenotrophomonas maltophilia, in accordance with the publication of Windhorst et al. (Journal of Biological Chemistry Vol. 277, No. 13: 11042-11049, 2002), GenBank™ accession number AJ291488, and

(4) Protease BPN′ as an example of a protease of the subtilisin type.

The sequence comparison yields the following identity values:

| Comparison of | with | % identity |
|---|---|---|
| SmP2 | StmPr2 | 97.1 |
| SmP2 | StmPr1 | 42.4 |
| SmP2 | BPN′ | 17.3 |

A comparison of SmP2 with a shortened StmPr1 sequence (467 amino acids long) in accordance with database entry AJ291488.2, which represents the putative mature protease, yielded an identity value of only 37.3%.

FIG. 2: Sequence comparison (alignment) of the amino acid sequences of the proteases according to the present invention in accordance with SEQ ID NO. 2, SEQ ID NO. 6, SEQ ID NO. 8, SEQ ID NO. 10, SEQ ID NO. 12, and SEQ ID NO. 14. Also indicated is the amino acid sequence of the protease StmPr2 (SEQ ID NO. 4) from Stenotrophomonas maltophilia in accordance with accession numbers AAP13815 and AY253983 (nucleic acid sequence and corresponding amino acid sequence) of the publicly accessible database of the NCBI.

1-17. (canceled)

18. An agent comprising an isolated protease that comprises an amino acid sequence that is at least 85% identical to the amino acid sequence indicated in SEQ ID NO:2, SEQ ID NO:6, SEQ ID NO:8, SEQ ID NO:10, or SEQ ID NO:12.

19. The agent according to claim 18, wherein the agent is a washing agent, hand washing agent, rinsing agent, hand dishwashing agent, automatic dishwashing agent, cleaning agent, dental-prosthesis care agent, contact-lens care agent, post-treatment rinsing agent, disinfecting agent, cosmetic agent, pharmaceutical agent, or an agent for the treatment of filter media, textiles, furs, paper, fleeces, or leather.

20. The agent according to claim 18 wherein the agent is a laundry washing agent or a dishwashing agent.

21. The agent according to claim 18 wherein the agent exists as a one-component system.

22. The agent according to claim 18 wherein the agent is divided into multiple components.

23. The agent according to claim 18 wherein the agent comprises from 2 μg to 20 mg of the protease per g of the agent.

24. The agent according to claim 18 wherein the agent comprises from 5 μg to 17.5 mg of the protease per g of the agent.

25. The agent according to claim 18 wherein the agent comprises from 20 μg to 15 mg of the protease per g of the agent.

26. The agent according to claim 18 wherein the agent comprises from 50 μg to 10 mg of the protease per g of the agent.

27. The agent according to claim 18 wherein the agent exists in solid form, in pasty form, or in liquid form.

28. The agent according to claim 18 wherein the agent is a pourable powder having a bulk weight of from 300 g/l to 1200 g/l.

29. The agent according to claim 18 wherein the agent is a pourable powder having a bulk weight of from 500 g/l to 900 g/l.

30. The agent according to claim 18 wherein the protease is encased with a substance that is impermeable to the protease at room temperature or in the absence of water.

31. The agent according to claim 18 wherein the agent contains one or more additional proteases, and/or one or more amylases, cellulases, hemicellulases, mannanases, tannases, xylanases, xanthanases, β-glucosidases, carrageenases, oxidases, oxidoreductases, or lipases.

32. An isolated protease comprising an amino acid sequence that is at least 97.2% identical to the amino acid sequence indicated in SEQ ID NO:2, is at least 93.7% identical to an the amino acid sequence indicated in SEQ ID NO:6, is at least 92.4% identical to the amino acid sequence indicated in SEQ ID NO:8, is at least 96.8% identical to the amino acid sequence indicated in SEQ ID NO:10, or is at least 92.4% identical to the amino acid sequence indicated in SEQ ID NO:12.

33. The protease of claim 32 comprising the amino acid sequence indicated in SEQ ID NO:2, SEQ ID NO:6, SEQ ID NO:8, SEQ ID NO:10, or SEQ ID NO:12

34. A method for cleaning textiles or hard surfaces comprising contacting the textiles or hard surfaces one or more times with 40 μg to 4 g per application of an isolated protease comprising an amino acid sequence that is at least 85% identical to the amino acid sequence indicated in SEQ ID NO:2, SEQ ID NO:6, SEQ ID NO:8, SEQ ID NO:10, or SEQ ID NO:12.

35. The method of claim 34 wherein the textiles or hard surfaces are contacted with 50 μg to 3 g of the protease per application.

36. The method of claim 34 wherein the textiles or hard surfaces are contacted with 100 μg to 2 g of the protease per application.

37. The method of claim 34 wherein the textiles or hard surfaces are contacted with 200 μg to 1 g of the protease per application.
